(19) United States
(12) Patent Application Publication
(10) Pub. No.: US 2004/0185043 A1
(43) Pub. Date: Sep. 23, 2004

(54) Hematopoietic stem cell chimerism to treat autoimmune disease

(76) Inventor: Suzanne T. Ildstad, Prospect, KY (US)

Correspondence Address: HOGAN & HARTSON LLP, ONE TABOR CENTER, SUITE 1500, 1200 SEVENTEENTH ST, DENVER, CO, 80202, US

(21) Appl. No.: 10/702,058

(22) Filed: Nov. 5, 2003

Related U.S. Application Data
(63) Continuation of application No. PCT/US02/14749, filed on May 9, 2002.
(63) Continuation of application No. 10/134,016, filed on Apr. 26, 2002.
(60) Provisional application No. 60/290,167, filed on May 9, 2001.
(60) Provisional application No. 60/290,166, filed on May 9, 2001.
(60) Provisional application No. 60/375,884, filed on Apr. 26, 2002.

(51) Int. Cl. A61K 45/00; A61K 39/395

(52) U.S. Cl. 424/140.1; 424/93.71

(57) ABSTRACT

The primary focus of this the present invention is to disclose a minimal conditioning approach to establish mixed chimerism to induce tolerance in recipients having autoimmune disease. Engraftment is multifactorial. Both donor and host factors influence engraftment as independent but complementary variables. By optimizing first the donor factors that influence outcome and then defining the recipient factors that resist engraftment. Chimerisms in recipients with minimum morbidity can be achieved, thus allowing for the prevention or treatment of autoimmune disease.

### CROSS-REFERENCE TO RELATED APPLICATIONS

This is a Continuation application of PCT/US02/14749, filed May 9, 2002, which claims priority to U.S. Provisional Application Serial No. 60/290,167 filed May 9, 2001, U.S. Provisional Application Serial No. 60/290,166 filed May 9, 2001, U.S. Provisional Application Serial No. 60/375,884 filed Apr. 26, 2002, and U.S. patent application Ser. No. 10/134,016, filed Apr. 26, 2002, each of which is incorporated herein by reference in its entirety.

### BACKGROUND OF THE INVENTION

1. Field of the Invention

This invention relates to novel methods relating to the reversal of the autoimmune process itself. Specifically, minimal conditioning strategies in combination with the clinical application of hematopoietic stem cell (HSC) will allow for new therapeutic approaches of bone marrow transplantation (BMT) to prevent and/or treat systemic autoimmune diseases.

2. Description of the Prior Art

Type I diabetes is a disease in which the insulin producing islet tissue is destroyed by the patient's own immune system. When the body attacks itself in this manner, it is called “autoimmunity.” Diabetes affects approximately 16 million people in the United States alone. It causes shorter life spans and also increased infant death rates. Over 40% of the people living with diabetes develop kidney failure in their lifetime. Other complications include blindness, nerve damage and hardening of the arteries. The discovery of insulin has prevented death from acute diabetes.

However, even with tight glucose control the systemic complications progress. A method to induce tolerance and reverse the autoimmunity would be of major benefit. Hematopoietic stem cell (HSC) transplantation leading to allogeneic chimerism has been proposed as one approach for the induction of transplantation tolerance and the treatment of autoimmune disorders. Implementation of clinical trials for treatment of autoimmune diseases such as type I diabetes has in part been impeded by the toxicity of conditioning procedures required to establish engraftment and the morbidity from graft versus host disease (GVHD).

There is strong evidence to support that a number of autoimmune diseases, including type I diabetes, systemic lupus erythematosus, rheumatoid arthritis, scleroderma, psoriasis, multiple sclerosis, and Crohn's colitis, are linked to the HSC. (Akizuki M., et al., Clinical Immunology and Immunopathology, 10:247 (1978); LaFace, D. M., et al., Diabetes, 38:894 (1989); Morton, J. I., et al., Proceed. Natl. Acad. Sci. USA, 71:2162 (1974); Nakamura, T., et al., Thymus, 7:151 (1985); Theofilopoulos, A. N., et al., Advances in Immunology, 37:269 (1985)). Bone marrow transplantation (BMT) from disease-prone donors transfers the autoimmune disease in mice, rats, and humans (Vialettes, B., et al., Diabetologia, 36:541 (1993); Carrier, E., et al., Current Opinion in Organ Transplantation, 5:343 (2000)). (Morton, J. I., et al., Proceed. Natl. Acad. Sciences of the USA, 71:2162 (1974); Ikehara, S., et al., Proceed. Natl. Acad Sciences of the USA, 87:8341 (1990); Serreze, D. V., et al., Diabetes, 37:252 (1988); Naji, A., et al., Annals of Surgery; 194:328 (1981).) Conversely, BMT from disease-resistant donors cures the autoimmune process (Marmont, A. M., et al., J. Rheumatol, 24:13 (1997); Burt, R. K., et al., Stem Cells, 17:366 (1999); Comi, G., et al., J. Neurol, 247:376 (2000); Burt, R. K., et al., Blood, 92:3505 (1998)). In 1974 Morton and Siegel reported that BMT cured the autoimmune process in NZB mice (Morton, J. I., et al., Transplantation, 27:133 (1979)). Similarly, fully allogeneic BMT (A→B) prevents the development of overt diabetes in NOD mice and BB rats (Naji, A., et al., Annals of Surgery, 194:328 (1981)), MRL/lpr and MRL/gld mice (Theofilopoulos, A. N., et al., J. Experimental Medicine, 162:1 (1985); Breban, M., et al., J. Experimental Medicine, 178:1607 (1993)) and histocompatibility antigens (HLA) B27 transgenic rats (Breban, M., et al., J. Experimental Medicine, 178:1607 (1993)). More recently, compelling evidence for reversal of autoimmunity in humans after HSC transplantation has emerged. Humans have been cured of type I diabetes early after onset while in the honeymoon period, rheumatoid arthritis, Systemic Lupus Erythematosus, psoriatic arthritis, Crohn's colitis, and multiple sclerosis after fully ablative allogeneic BMT for leukemia (Burt, R. K., et al., Stem Cells, 17:366 (1999); Comi, G., et al., J Neurol, 247:376 (2000); Baldwin, J. L., et al., Arthritis and Rheumatism, 20:1043 (1977); Eedy, D. J., et al., British Medical Journal, 300:908 (1990); Jacobs, P., et al., Bone Marrow Transplantation, 1986:237 (1986); Liu, Y., et al., Bone Marrow Transplantation, 9:31 (1992); Lowenthal, R. M., J.f Rheumatology, 20:137 (1993); Marmont, A. M., et al., Bone Marrow Transplantation, 9:1 (1992); Rose, N. R., et al., Immunology Today, 14:426 (1993); Roubenoff, R., et al., Arthritis and Rheumatism, 30:1187 (1987)). Although the morbidity and mortality associated with fully ablative BMT could not be justified, this observation may allow a promising new approach if the risk/benefit ratio for BMT could be reduced significantly.

Chimerism is a state in which two separate immune systems can live in one body. In order for the bone marrow to “take” or “engraft,” the recipient receives a dose of irradiation before the bone marrow transplant to make space for the new bone marrow. If the donor and recipient bone marrow co-exist (the recipient will not “reject” the donor bone marrow) a state of mixed bone marrow chimerism is achieved and the autoimmune process should be reversed. The establishment of mixed bone marrow chimerism has been shown to reverse the autoimmune process and to prevent the onset of diabetes in laboratory animals.

Ildstad, et al., reported that stable mixed allogeneic (A+B→B) HSC chimerism could be achieved by transplantation of a mixture of T cell depleted (TCD) syngeneic plus TCD allogeneic marrow into fully conditioned recipients (950 cGy) (Ildstad, S. T., et al., Nature, 307:168 (1984)). Recipients exhibit durable multilineage chimerism and are tolerant to donor-specific islet Lim, H., et al., Transplantation, 57:592 (1994), cardiac Colson, Y., et al., Transplantation, 60:971 (1995), and skin allografts Ildstad, S. T., et al., Nature, 307:168 (1984). Mixed chimeras exhibit superior immunocompetence for primary immune responses due to the presence of recipient-derived antigen presenting cells (APC) (Ruedi, E., et al., Cellular Immunology, 121:185 (1989); Ildstad, S. T., et al., J. Medicine, 160:1820 (1984); Colson, Y. L., et al., J Immunology, 160:3790 (1998)), and immunocompetence to respond to major histocompatibility complex (MHC)-disparate third party alloantigen (Ildstad, S. T., et al., J. Experimental Medicine, 162:231 (1985)). Moreover, mixed chimerism can be established by partially ablative conditioning followed by allogeneic BMT, thereby avoiding the morbidity of full ablation. If total body irradiation (TBI) alone is utilized, 650 cGy is required to establish mixed chimerism in 100% of recipients (Colson, Y. L., et al., J. Immunology, 155:4179 (1995); Colson, Y. L., et al., J. Immunology; 157:2820 (1996)). Preconditioning of the host with antilymphocyte globulin (ALG) plus cyclophosphamide (200 mg/kg intraperitoneally [IP]) on day +2 (Colson, Y. L., et al., J. Immunology, 155:4179 (1995); Colson, Y. L., et al., J. Immunology, 157:2820 (1996)) or anti-CD8 mAb (Exner, B. G., et al., Surgery, 122:221 (1997)) reduces the TBI dose to 200 cGy. Doubling of the marrow cell dose allows engraftment with only 100 cGy of TBI (Colson, Y. L., et al., J. Immunology, 155:4179 (1995); Colson, Y. L., et al., J. Immunology, 157:2820 (1996)). Removal of graft facilitating cells (FC) results in a greater requirement for conditioning.

The FC is a CD8⁺/TCR⁻ cell in bone marrow that facilitates engraftment of highly purified HSC in allogeneic recipients. However, if FC are also present, T cells increase the level of donor chimerism. FC are critical to long-term survival of HSC, while CD8⁺ T cells are only supplemental in that they provide space-making potential.

Schuchert, et al., confirmed that CD3ε⁺/TCR⁻/CD8⁺ FC facilitated HSC engraftment and reported that marrow from TCR-β knock out mice does not contain functional FC, suggesting a pre-T cell lineage commitment (Colson, Y. L., et al., Nature Medicine, Submitted: 1999). Moreover, a unique 33 kilodalton glycoprotein associated with the CD3ε chain correlates with the functional ability of FC to enhance allogeneic HSC engraftment in vivo. An editorial accompanying the report described FC as a cell that may have a significant impact upon bone marrow and solid organ transplantation. The term facilitating cell has been incorporated into the stem cell vernacular and the cell population continues to be further characterized and defined. However, the overwhelming consensus is that CD8⁺/TCR⁻ bone marrow-derived cells mediate an important and highly reproducible effect on stem cell engraftment in allogeneic recipients.

Pancreas and islet cell transplantation. Transplantation of whole pancreas or purified insulin-producing islets is the preferred approach to achieve glucose homeostasis. Normoglycemia slows the progression of complications, especially the nephropathy (Orloff, M. J., et al., Surgery, 1990:179 (1990); Gotoh, M., et al., Transplantation, 208:475 (1988); Sollinger, H. W., et al., Annals of Surgery, 208:475 (1988); Sutherland, D. R., et al., UCLA Press, (1988); Kennedy, W. R., et al., N. Engl. J. Med., 322:1031 (1990); Bilous, R. W., et al., N. Engl. J. Med., 321:80 (1989); Ramsay, R. C., et al., N. Engl. J. Med., 318:208 (1988); Petersen, M. R., et al., Ophthalmology, 97:496 (1990)). Transplantation of insulin-producing tissue has the added advantage that the hypoglycemia associated with exogenous insulin administration can be avoided. Islets are regulated in function and can efficiently maintain normoglycemia. However, rejection remains a major limitation in pancreas and islet cell transplantation. Only 65% of pancreas transplants function at five years, in spite of conventional immunosuppressive agents (Gruessner, A. C., et al., Clinical Transplantation, 4:45 (1997)). Islet allografts have the advantage that they can be transplanted nonoperatively transcutaneously via the portal vein. However, although technically feasible, transplantation of purified islets have experienced limited success. In over 400 islet transplants performed, few patients have been rendered insulin-independent (Hering, B. J., et al., Graft, 2:12 (1999); Sutherland, D. E., et al., Transplant Proceedings, 30:1940 (1998)). Recently, it was reported that if prednisone is avoided and multiple donors for pancreas are utilized, patients can be rendered insulin-independent following islet transplantation (Shapiro, A. M., et al., N. Engl. J. Med., 343:230 (2000)). However, follow-up in these patients is still short and the requirement for at least 2 donors per recipient would be a severe limitation in the long term due to potential demand versus supply of cadaveric donors. A method to induce tolerance to the transplanted tissue would be a major advantage.

Bone marrow chimerism induces donor-specific tolerance to islet allografts and xenografts. Bone marrow transplantation induces tolerance to solid organ and cellular transplants (Acholonu, I., et al., Current Opinion in Organ Transplantation, 4:196 (1999)). The first association between HSC chimerism (macrochimerism) was made by Owen in 1945, who reported that freemartin cattle were red blood cell chimeras (Owen, R. D., et al., Science, 102:400 (1945)). Owen hypothesized that exchange of HSC occurred in utero in the placenta between the two genetically disparate cattle twins. Contemporaneously, Billingham, et al., reported that freemartin cattle accepted reciprocal skin grafts from the other genetically disparate sibling donor (Billingham, R. E., et al., Heredity, 6:201 (1952)). Aware of Owen's report, a neonatal mouse model to define what tissues were critical for the active transfer of tolerance from one donor to another genetically disparate one was established. Hematopoietic-derived tissues (HDT) (splenocytes or bone marrow) transplanted into unmanipulated neonatal mice actively transferred donor-specific tolerance to skin allografts (Billingham, R. E., et al., Nature, 172:606 (1953)). When an attempt was made to establish a similar model in adult mouse recipients, chimerism could not be established, leading to the recognition that conditioning to make space for the newly infused marrow was required in adult recipients.

Bone marrow chimerism induces self-tolerance and reverses autoimmunity. Significant progress has been made in understanding the underlying mechanism for autoimmune diabetes through study of the NOD mouse. A seminal observation that has opened a new strategy for the treatment of autoimmune diabetes is that HSC chimerism reverses the active autoimmune process (Naji, A., et al., Analytical and functional studies on the T cells of untreated and immunologically tolerant diabetes-prone BB rats).

Evidence is accumulating that a number of autoimmune diseases, including systemic lupus erythematosus (SLE), rheumatoid arthritis (RA), scleroderma, multiple sclerosis (MS), colitis and spondyloarthropathy, as well as type I diabetes, are linked to the HSC itself rather than its derivatives (LaFace, D. M., et al., Diabetes, 38:894 (1989); Morton, J. I., et al., Proceed. Natl. Acad. Sciences of the USA, 71:2162 (1974); Akizuki, M., et al., Clinical Immunology and Immunopathology, 10:247 (1978); Nakamura, T., et al., Thymus, 7:151 (1985); Theofilopoulos, A. N., et al., Advances in Immunology, 37:269 (1985)). Bone marrow from disease-prone donors transfers the disease process, and conversely, transplantation of bone marrow from disease-resistant donors into autoimmune recipients reverses the autoimmunity (Li, H., et al., J. Immunology, 156:380 (1996); Morton, J. I., et al., Proceed. Natl. Acad. Sciences of the USA, 71:2162 (1974); Morton, J. I., et al., Transplantation, 27:133 (1979); Ikehara, S., et al., Proceed. Natl. Acad. Sciences of the USA 82:7743 (1985)). This observation has been confirmed in a number of animal models and in humans. BMT has therefore been suggested as a potential strategy to interrupt the autoimmune process.

In 1974, Morton and Siegel showed that autoimmunity in NZB mice could be transferred by hematopoietic cells into lethally irradiated disease-resistant recipients (Morton, J. I., et al., Proceed. Natl. Acad. Sciences of the USA, 71:2162 (1974)). Conversely, BMT from disease-resistant donors prevented autoimmunity in NZB mice (Morton, J. I., et al., Transplantation, 27:133 (1979)). This was confirmed in 1977 by DeHeer and Edgington (DeHeer, D. H., J. Immunology, 118:1858 (1977)). Subsequently, disease prevention was reported in NOD mice (Ikehara, S., et al., Proceed. Natl. Acad. Sciences of the USA, 82:7743 (1985)) and BB rats (Naji, A., et al., Annals of Surgery, 194:328 (1981)) that were fully conditioned and transplanted with bone marrow from disease-resistant donors. Transfer of autoimmunity by BMT has also been achieved in MRL/lpr and BXSB mice, as well as HLAB27 transgenic rats (Theofilopoulos, A. N., et al., J. Experimental Medicine, 162:1 (1985); Breban, M., et al., J. Experimental Medicine, 178:1607 (1993)). Unmodified marrow was administered in the early studies, implicating T cells within the donor inoculum as potential mediators of the effect. Splenocytes or lymph node lymphocytes did not transfer disease, pointing to a role for the HSC itself. When marrow from nude mice or TCD marrow from normal allogeneic donors achieved reversal of autoimmunity in disease-prone recipients, including models for type I diabetes, data in support of a role for the HSC, rather than its progeny (T cells) emerged (LaFace, D. M., et al., Diabetes, 38:894 (1989); Ikehara, S., et al., Proceed. Natl. Acad. Sciences of the USA, 82:7743 (1985); Naji, A., et al., Annals of Surgery, 194:328 (1981); Theofilopoulos, A. N., et al., J. Medicine, 162:1 (1985); Breban, M., et al., J. Experimental Medicine, 178:1607 (1993); Serreze, D. V., et al., Diabetes, 37:252 (1988); Ikehara, S., et al., Proceed. Natl. Acad. Sciences of the USA, 82:2483 (1985); Ikehara, S., et al., Immunology, 86:3306 (1989); Wicker, L. S., J. Experimental Medicine, 167:1801 (1988)). Finally, transplantation of purified HSC alone from NOD mice into disease-resistant recipients transfers the systemic autoimmune state (Ikehara, S., et al., Proceed. Natl. Acad. Sciences of the USA, 87:8341 (1990)). The thymus and environmental factors appear to act only as accelerating factors that contribute to the underlying process. Van Bekkum recently suggested that autoimmune diseases should be classified as HSC defects and differentiated from defects in stromal cells (i.e., diGeorge athymia) that are not treatable by BMT (Van Bekkum, D. W., et al., Bone Marrow Transplantation, 11:183 (1993)).

After the recognition in animal models that autoimmunity can be transferred and cured by BMT, case reports have confirmed a similar observation in humans. Autoimmunity has been transferred by BMT to disease-free human recipients (Holland, J., et al., J. Clinical Endocrinology & Metabolism, 72:837 (1991); Lampeter, E. F., et al., Lancet, 341:1243 (1993); Vialettes, B., et al., Diabetologia, 36:541 (1993)). Conversely, BMT from disease-resistant donors reverses the autoimmune process (Marmont, A. M., et al., Forum, 5:24 (1995)). A number of patients with leukemia and other malignancy with underlying autoimmune disease have undergone BMT. Seven patients with RA who received an allogeneic BMT from disease-free HLA-identical siblings experienced a dramatic reduction in severity of the disease. In six of seven patients, complete and long-lasting remission was achieved (Lowenthal, R. M, et al., J. Rheumatology, 20:137 (1993); Baldwin, J. L., et al., Arthritis and Rheumatism, 20:1043 (1977); Jacobs, P., et al., Bone Marrow Transplantation, 1986:237 (1986); Jacobs, P., et al., Bone Marrow Transplantation, 1986:237 (1986); Rose, N. R., et al., Immunology Today, 14:426 (1993); Marmont, A. M., et al., Bone Marrow Transplantation, 9:1 (1992)). Other autoimmune diseases including Crohn's colitis (Liu, Y., et al., Bone Marrow Transplantation, 9:31 (1992)), psoriatic arthritis (Liu, Y., et al., Bone Marrow Transplantation, 9:31 (1992); Fadok, V. A., et al., J. Immunol, 149:4029 (1992)), MS, lupus, and type I diabetes in the honeymoon period (Tyndall, A., et al., J. Rheumatology Supplement, 48:94 (1997)) have been put into remission following BMT. Because life-threatening autoimmune diseases are not controlled by conventional immunosuppressive therapy, fully ablative conditioning followed by allogeneic BMT is being tested in clinical trials as a therapeutic strategy in patients with late-stage disease. The field is moving ahead so rapidly that BMT for autoimmunity is the topic of a major annual international meeting. Animal models such as the NOD mouse have proven to be effective tools to explore mechanistic questions in refining this approach for clinical application.

A number of factors are hypothesized to contribute to the limited success in islet and pancreas transplant survival; (1) alloreactivity; (2) autoreactivity; (3) islet cell dose. There is still a need, therefore, to identify the critical factors, and that the requirement for increasing numbers of islet cells is in part due to the ongoing chronic destruction of transplanted islet-bearing tissue from alloreactivity and autoimmunity in the diabetic recipient.

Therefore, there is a need to develop an approach to optimize chimerism in recipients having autoimmune diseases. The focus of the present invention is to develop a safe method to induce donor-specific transplantation tolerance to islet cell allografts would offer the following potential advantages: (1) the requirement for nonspecific immunosuppressive agents would be avoided; (2) the autoimmune process would be halted; and (3) glucose homeostasis could be achieved long term. When clinically applied, this treatment could dramatically increase the life expectancy and improve the quality of life of millions of people afflicted with diabetes. This procedure may even lead to our ability to cure the autoimmune process before the development of life-threatening complications.

There is further a need for strategies to partially condition a recipient to achieve a low level of donor chimerism and tolerance could be pursued with the advantage that they could avoid the risk of fully ablative conditioning. Donor-specific islet allografts are permanently accepted by mixed chimeras, while MHC-disparate allografts are promptly rejected. The tolerance associated with chimerism is so robust that mixed xenogeneic chimeras (mouse+rat→mouse) also permanently accept donor-specific islet xenografts but promptly reject MHC-disparate rat xenografts and mouse allografts (Kaufman, C. L., et al., J. Immunology, 158:2435 (1997)). (Li, H., et al., J. Immunology, 156:380 (1996); Kaufman, C. L., et al., Blood, 84:2436 (1994).) Moreover, recipients who are 1% donor are just as tolerant as those that are 99% donor.

### SUMMARY OF THE INVENTION

Accordingly, it is an object of this invention to induce tolerance to islet allografts after insulin-dependence has developed using HSC chimerisms.

It is a further object of the present invention to interrupt the autoimmune process by inducing self-tolerance before the development of irreversible terminal complications.

A further object of the present invention is to define the role of FC in establishing chimerism and tolerance in NOD mice, and, furthermore to define the role of CD8⁺/TCR⁻, CD8⁺/TCR⁺, and γδ-T cells as FC in HSC engraftment. As a result, the critical elements to optimize the composition of the donor marrow graft and allow durable HSC survival in NOD recipients will be defined.

Another object of this invention is to provide minimal conditioning strategies to establish mixed chimerism in recipients suffering from an autoimmune disease by defining the mechanism underlying the hematopoietic abnormalities in recipients having autoimmune diseases.

Another object of this invention is to characterize which cells in the NOD microenvironment influence engraftment, thus allowing one to identify the critical cells to be removed from the NOD microenvironment or immunosuppressed to allow engraftment of disease-resistant marrow.

A further object is to refine a partial conditioning approach with the goal to reduce or eliminate the requirement for irradiation by selectively removing specific cells from the host microenvironment to make space for durable engraftment of normal HSC.

Additional objects, advantages, and novel features of this invention shall be set forth in part in the description and examples that follow, and in part will become apparent to those skilled in the art upon examination of the following or may be learned by the practice of the invention. The objects and the advantages of the invention may be realized and attained by means of the instrumentalities and in combinations particularly pointed out in the appended claims.

### BRIEF DESCRIPTION OF THE FIGURES

The accompanying drawings, which are incorporated herein and form a part of the specification, illustrate preferred embodiments of the present invention, and together with the description, serve to explain the principles of the invention.

FIG. 1 is a bar graph illustrating the level of Fas lymphocytes in bone marrow, spleen, and thymus tissues from NOD mice and BR mice.

FIG. 2 is a bar graph illustrating the percent CD4⁺ T cells in bone marrow, spleen, and thymus tissues from NOD mice and BR mice.

FIG. 3 is a graph showing the percent survival versus days post-transplant in recipients of FC (circles), MHC-disparate recipients (squares) and for donor and recipient matched at the MHC but disparate for minor antigens (triangles).

FIG. 4 is a bar graph representing the percent positive cells for DX5, 5E6, 2B4 and NK/T subsets of NK cells for NOD and BR recipients.

FIG. 5 is a graph showing percent engraftment verses total body irradiation (TBI) in NOD recipients pretreated with anti-CD40L (squares), anti-CTLA-4 (triangles) and in NOD recipients without pretreatment (diamonds).

FIG. 6 is a bar graph representing the percent level of engraftment one month after bone marrow transplant (BMT) at various doses of total body irradiation (TBI).

FIG. 7 is a bar graph representing the percent level of engraftment four months after bone marrow transplant (BMT) at various doses of total body irradiation (TBI).

FIG. 8 is a graph showing the percent NK cell viability after 20 hours versus total body irradiation (TBI) dose in cells from NOD mice (triangles) and cells from BR mice (squares).

FIG. 9 is a graph showing percent [³H]-Thymidine incorporation verses concentration of anti-CD3 monoclonal antibody for BR, NOD and NON control mice.

FIG. 10 is a bar graph representing the percent positive cells in HAS^{hi} gate for AA4.1 and Ly6C cells.

FIG. 11 shows the results of a Western blot illustrating the expression of Bas, FasL and Bcl-2 genes in marrow.

FIG. 12 shows the results of a RT-PCR illustrating the expression of Bas, FasL and Bcl-2 genes in spenocytes.

FIG. 13 illustrates the expression of FasL at the protein level in chimeras.

FIG. 14 is a bar graph showing the percent positive FC cells from B10.BR mice bone marrow cells and from NOD mice bone marrow cells.

### DETAILED DESCRIPTION OF THE INVENTION

The primary focus of the present invention is to disclose a minimal conditioning approach to establish mixed chimerism to induce tolerance in recipients having autoimmune diseases. The Examples disclosed below utilize the NOD mouse model for type I diabetes. For clarity of discussion, the specific procedures and methods described herein are exemplified using a murine model; they are merely illustrative for the practice of the invention. Analogous procedures and techniques are equally applicable to all mammalian species, including human subjects. The NOD mouse recipient is extremely sensitive to failure of engraftment when the donor marrow is T cell depleted. In addition, much higher numbers of unmodified bone marrow cells are required for durable engraftment in NOD mice compared to normal mice as recipients. Finally, although NOD mice initially engraft as mixed chimeras, the percentage of donor chimerism becomes increasingly high over the next four to six months until it approaches 100%. Taken together, these observations indicate that the NOD marrow microenvironment is abnormal and relatively alloresistant to engraftment. The facilitating cell (FC) is a CD8⁺/TCR⁻/CD3⁺ cell in bone marrow that enables engraftment of highly purified HSC in allgeneic recipients without causing GVHD. Conventional T cell depletion (TCD) approaches would also remove FC. However, it should be understood that similar procedures and compositions could be used in any mammal afflicted with diabetes, particularly humans. Engraftment is multifactorial. Both donor and host factors influence engraftment as independent but complementary variables. By optimizing first the donor factors that influence outcome and then defining the recipient factors that resist engraftment, we hypothesize that we will achieve chimerism in NOD recipients with minimum morbidity.

By combining cytoreductive with immunosuppressive agents with nonoverlapping mechanisms of action and optimizing marrow composition, one can obtain HSC chimerism with minimal conditioning in normal recipients.

Censoring of autoimmune cells occurs by apoptosis when the developing cells encounter self-antigen and respond too strongly. Centrally this is regulated by genes in the Bcl family. In the periphery, chronically activated cells are usually censored by Activation-Induced Cell Death (AICD), a pathway mediated via interaction of Fas:Fas Ligand (FasL). Cells from non-obese diabetic (NOD) mice are relatively resistant to apoptosis, relatively radioresistant, and in a chronically activated state. It is believed that HSC chimerism restores normal apoptotic machinery lacking in NOD mice. The NOD defect may be at the level of cell surface signaling (e.g., Fas-FasL caspase pathway), or of intracellular regulation of mitochondrial function (e.g., Bcl-2 and p53 protein families.

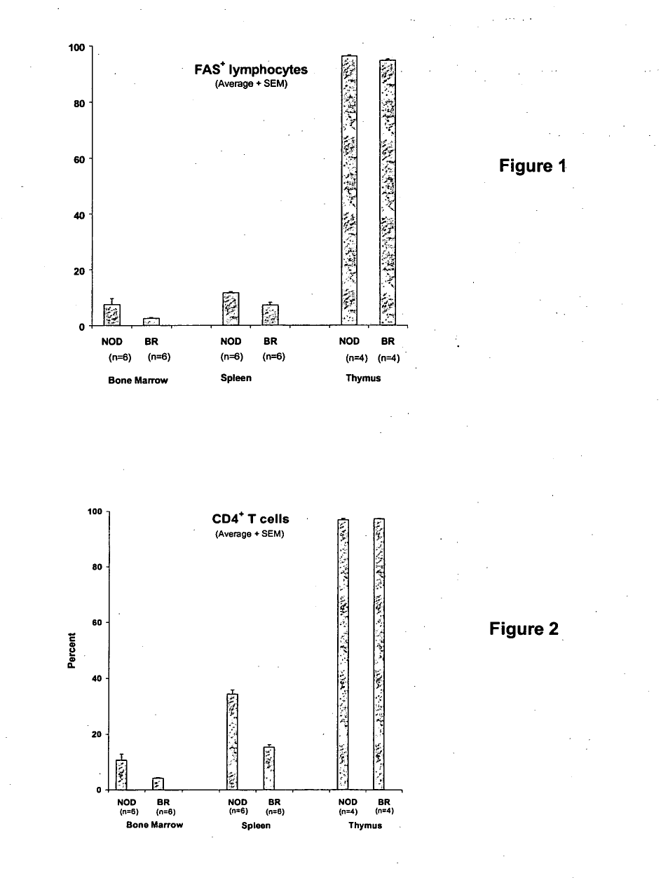

Self-reactive T cells are censored during their development by clonal deletion (Akizuki, M., et al., Clinical Immunology and Immunopathology, 10:247 (1978); Nakamura, T., et al., Thymus, 7:151 (1985); Theofilopoulos, A. N., et al., Advances in Immunology, 37:269 (1985)). APC, especially bone marrow derived dendritic cells, are the most potent deleting ligand (Morton, J. I., et al., Transplantation, 27:133 (1979); Ikehara, S., et al., Proceed. Natl. Acad. Sciences of the USA, 82:7743 (1985); DeHeer, D. H., J Immunology, 118:1858 (1977); Naji, A., et al., Annals of Surgery, 194:328 (1981); Theofilopoulos, A. N., et al., J. Experimental Medicine, 162:1 (1985); Breban, M., et al., J. Experimental Medicine, 178:1607 (1993)). APC from NOD mice and humans with diabetes (Serreze, D. V., et al., Diabetes, 37:252 (1988)) exhibit an impaired ability to mediate clonal deletion due to a generalized defect in protein kinase C activation (Ikehara, S., et al., Proceed. Natl. Acad. Sciences of the USA, 82:2483 (1985)). It is believed that mixed chimerism restores clonal deletion in NOD mice by providing normal APC to restore a defective pathway for apoptosis. NOD mice produce abnormal Fas Ligand (FasL). Although NOD mice produce FasL, much larger quantities are required to activate the Fas Receptor (Ikehara, S., et al., Immunology, 86:3306 (1989)). In preliminary analyses the level of expression of Fas Receptor in NOD mice has been evaluated. The level of Fas expression in the NOD lymphocytes is significantly greater in bone marrow and spleen (FIG. 1). NOD mice contain more CD4⁺ T cells in their bone marrow and spleen compared to BR mice (FIG. 2). It will be informative to compare the level of FasL and Fas in NOD mixed chimeras versus unmanipulated NOD controls to determine whether the normal bone marrow-derived cells restore effective apoptosis via production of normally functional FasL.

The FC could induce tolerance and restore self-tolerance in NOD mice by: (1) acting as a tolerance-inducing cell that migrates to the thymus to restore clonal deletion directly, or (2) maintain pluripotency and survival of the HSC which produces tolerizing dendritic cells and newly developing T cells to re-establish normal censoring (deletion) of autoreactive T cells. Goodnow et al. demonstrated that transgenic mice capable of producing autoimmune B cells did so only if they were forced to. If normal marrow was also present, as in mixed chimerism, competitive censoring of the autoimmune B cells occurred via a Bcl-2 dependent pathway, resulting in production of only normal B cells (Wicker, L. S., J Experimental Medicine, 167:1801 (1988); Ikehara, S., et al., Proceed. Natl. Acad. Sciences of the USA, 87:8341 (1990)). When Bcl-2 transgenic mice were utilized as the marrow donor the autoimmune process was not censored, demonstrating a critical mechanistic role for Bcl-2 in the autoimmune process. NOD mice could have a defective Bcl-2 process for clonal deletion. In NOD mice, although mixed chimerism can be established transiently (months 1-4) the percentage of donor multilineage chimerism changes to ≧95% donor by month 6 (Li, H., et al., Transplantation, 57:592 (1994); Van Bekkum, D. W., et al., Bone Marrow Transplantation, 11:183 (1993)). Consequently it is believed that the censoring of endogenous autoimmune cells is restored by BMT.

The present invention is achieved by determining the mechanism by which mixed chimerism reverses autoimmune diabetes. An understanding of the mechanism by which mixed chimerism reverses the autoimmunity will allow novel approaches to optimize the outcome to interrupt the autoimmune process. The primary focus of the experimental portion of the present invention is to develop a minimal conditioning approach to establish mixed chimerism in NOD mice by defining the components in donor bone marrow that are critical to engraftment of HSC and to optimize the effect. Consequently, these approaches can be applied to human subjects in a clinical setting.

Type I diabetes is the initial focus of the present invention when we applied mixed chimerism to induce tolerance to islet allografts. However, other autoimmune diseases such as but not limited to lupus, sleroderma, rheumatoid arthritis, multiple sclerosis, psoriasis, Crohn's Disease, colotis, anemia, and sickle cell may be prevented or treated by the same methodology. First normal disease-resistant B10.BR mice as recipients are used. Mice conditioned with fully ablative (950 cGy TB) conditioning were transplanted with allogeneic (Marmont, A. M., et al., Bone Marrow Transplantation, 9:1 (1992)) or xenogeneic (Hering, B. J., et al., Graft, 2:12 (1999)) islet grafts after mixed chimerism was established. Mice had been previously rendered diabetic using streptozotocin. Donor-specific islet grafts were permanently accepted while MHC-disparate grafts were promptly rejected. The islet grafts were able to maintain glucose homeostasis and respond to a glucose challenge (Liu, Y., et al., Bone Marrow Transplantation, 9:31 (1992); Fadok, V. A., et al., J. Immunol, 149:4029 (1992); Tyndall, A., et al., J. Rheumatology Supplement, 48:94 (1997); Li, H., et al., Transplant Proc, 31:640 (1999)). Moreover, the grafts were accepted when placed simultaneously with the marrow infusion (Hering, B. J., et al., Graft, 2:12 (1999); Marmont, A. M., et al., Bone Marrow Transplantation, 9:1 (1992); Cyster, J. G., et al., Nature, 371:389 (1994)) or sequentially after chimerism was established (Hering, B. J., et al., Graft, 2:12 (1999)). Full ablation was not essential to islet graft tolerance. Partially conditioned mice also accepted donor-specific islet allografts if they were rendered chimeric.

HSC Chimerism Reverses Autoimmunity in NOD Mice. The induction of mixed chimerism during the active autoimmune process at 12 weeks prior to the onset of overt diabetes completely reversed the systemic autoimmunity. While partially conditioned NOD controls that did not receive a marrow transplant developed insulin-dependent diabetes with the predicted frequency (80% by 12 months), none of the chimeras developed diabetes (Cyster, J. G., et al., Nature, 371:389 (1994); Li, H., et al., Surgery, 118:192 (1995)). Therefore, it was HSC chimerism and not the conditioning that reversed the autoimmune process. The mechanism for this effect has not yet been defined. However, as the mechanism responsible for reversal of the autoimmune process is defined, new strategies to potentiate the effect will emerge. The present invention draws on the belief that mixed chimerism restores the normal process for censoring potentially autoreactive cells by restoring defective apoptotic machinery.

HSC chimerism induces auto and allotolerance in NOD mice. After establishing that mixed chimerism induces tolerance in normal mice, the NOD mouse model for type I diabetes was used. Mixed chimerism induces donor-specific tolerance to islet allografts in diabetic NOD mice. Mice were conditioned with 950 cGy TBI and transplanted with a mixture of TCD syngeneic marrow plus untreated allogeneic donor marrow. The islets are regulated in function, even in response to a glucose challenge. Moreover, animals with a low level of donor chimerism at month one through four were just as tolerant as those with higher levels of chimerism. There was no evidence for recurrent insulitis in the newly transplanted islets. It appears that the autoimmune process was being reversed by chimerism. When twelve-week old NOD mice with active autoimmunity were transplanted with a mixture of syngeneic plus allogeneic marrow the autoimmune insulitis was reversed and they did not progress to develop full-blown diabetes (Kaufman, C. L., et al., J. Immunology, 158:2435 (1997)). Similarly, recipients that were partially conditioned and transplanted did not develop diabetes (Li, H., et al., J. Immunology, 156:380 (1996)) while conditioned but untransplanted controls did. Another group has now shown that the endogenous islets recover after establishment of allogeneic HSC Chimerism (Li, H., et al., Transplant Proc, 31:640 (1999)). Mixed chimerism has subsequently been demonstrated to cure other autoimmune disease states as well. MRL/gld mice defective in production of FasL are cured by induction of mixed Chimerism (Cyster, J. G., et al., Nature, 371:389 (1994)). Mice that develop autoimmune nephritis are also cured after partial conditioning and transplantation of allogeneic marrow to establish mixed chimerism (Wang, B., et al., Proc Natl. Acad. Sci. USA, 96:3012 (1999)).

The mechanism for tolerance induction by mixed HSC chimerism is by clonal deletion. The T cell repertoire is shaped by positive and negative selection events in the thymus (Marrack, P., et al., Cell, 53:627 (1988)). The final result is the mature T cell repertoire. The HSC produces two lineages that are critical to clonal deletion: pre-T cells and dendritic cells (DC). At least 5 subpopulations of DC exist, some of which are tolerogenic and others immunogenic (Pulendran, B., et al., Proc. Natl. Acad. Sci. USA, 96:1036 (1999)). Lymphoid dendritic cells (LDC) are the tolerogenic form. Only certain DC reside in the thymus. Pre-T cells that migrate to the thymus for further development undergo a number of genetically programmed developmental stages. During the state of intermediate maturity (CD8⁺/CD4+/TCR⁺) they are deleted from the repertoire if they respond too strongly against self- (or donor) antigen. DC provide the most efficient deleting ligand (Delaney, J. R., et al., Proc. Natl. Acad. Sci. USA, 95:5235 (1998)). Mixed chimerism therefore provides the most efficient conditions for deletional tolerance since both syngeneic and allogeneic donor DC are present. As a result, reciprocal co-tolerance to host as well as donor alloantigen are present in mixed chimeras.

Donor and host variables influence engraftment. The morbidity and mortality associated with fully ablative, conventional BMT could not be justified in clinical protocols to induce tolerance in type I diabetes. Engraftment is influenced by a number of variables that are interdependent and complementary. Donor factors as well as host factors influence outcome with respect to engraftment. Cell dose as well as the composition of the donor marrow profoundly influence engraftment. If very large numbers of donor bone marrow cells (BMC) are infused into genetically identical recipients, chimerism can be achieved without conditioning (Wu, D. D., et al., Hematology, 21:251 (1993); Stewart, F. M., Blood, 81:2566 (1993); Saxe, D. F., et al., Experimental Hematology, 12:277 (1984); Nilsson, S. K., et al., Blood, 89:4013 (1997); Santos, G. W., et al., N. Engl. J. Med., 309:1347 (1983)). Conversely, if the marrow is T cell depleted, significantly more conditioning is required (Vallera, D. A., et al., Transplantation, 47:751 (1989)). In addition, the approach for conditioning of the recipient to permit engraftment also influences outcome. If the composition of the marrow is enhanced, engraftment can be achieved with lesser conditioning. Conversely, more conditioning is required if T cell depletion of donor marrow is performed. A number of lessons from normal mice have contributed significantly to progress in the potential clinical application of mixed chimerism to induce tolerance in diabetes.

Donor factors that influence engraftment: Influence of the composition of the marrow on engraftment. The first report of a dichotomy between engraftment of purified HSC in MHC-disparate versus MHC-matched recipients was authored by El-Badri and Good, who hypothesized that purified HSC were metabolically inactive and could not come into cycle rapidly enough to effect engraftment (El-Badri, N. S., et al., Proceed. Natl. Acad. Sci. USA, 90:6681 (1993)). When they co-administered TCD syngeneic marrow with purified allogeneic HSC, they discovered that if the donor and recipient were MHC-congeneic, regardless of the minor antigens, the HSC engrafted readily. In striking contrast, if donor and recipient were MHC-disparate, engraftment of syngeneic but not allogeneic HSC resulted. However, when unmodified marrow is administered, physiologic numbers of allogeneic HSC engraft readily (Ildstad, S. T., et al., Nature, 307:168 (1984); El-Badri, N. S., et al., Proceed. Natl. Acad. Sci. USA, 90:6681 (1993)). They hypothesized that an accessory cell in marrow was required for HSC to engraft in MHC-disparate recipients.

It has been discovered that class I K are essential molecules for engraftment of allogeneic hematopoietic stem cells (HSC), since disparate at major histocompatibility complex (MHC) class I K locus between donor and recipient, impaired engraftment results. Conversely, with matching at class IK, successful engraftment was achieved. It was further discovered that facilitating cells (FC) are critical for engraftment of purified HSC in allogeneic recipients, since 100% animals of FC plus HSC exhibited durable mixed chimerism and long-term survival. When FC and HSC are matched at the class I K locus, FC exhibit a greater ability to facilitate engraftment of allogeneic HSC, suggesting that MHC class I K is an important molecule involved in the direct interaction between FC and HSC. The data discussed below in detail provide the first evidence that MHC class I K is an important molecule to influence engraftment of allogeneic HSC.

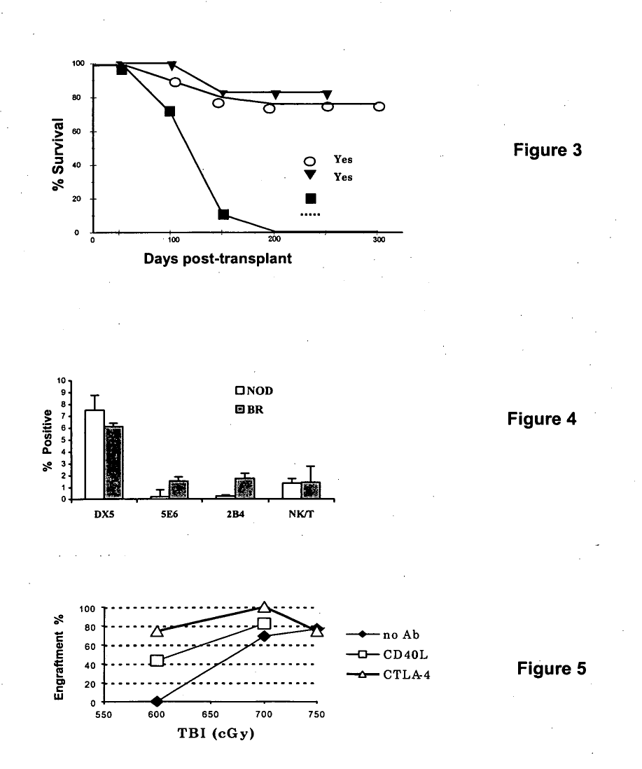

The present invention contemporaneously confirms these data using different strain combinations and a different approach for purifying HSC. While 1,000 highly purified HSC(C-kit⁺/Sca-1⁺/lin⁻) reliably rescue syngeneic recipients from radiation-induced aplasia (◯-◯), up to two logs more HSC do not readily engraft in MHC-disparate recipients (▪-▪) (FIG. 3) (Kaufman, C. L., et al., Blood, 84:2436 (1994)). If the donor and recipient are matched at the MHC but disparate for minor antigens (B10.BR and AKR), engraftment also occurs readily (▾-▾) (FIG. 3). The Stanford group reported that this barrier to engraftment of purified HSC can be overcome if up to 100 times more HSC are administered to fully ablated allogeneic recipients (Shizuru, J. A., et al., Biology of Blood and Marrow Transplantation, 2:3 (1996)). However, the caveat that a few FC:HSC doublets that slipped through as contaminants during the sorting process was not excluded (Shizuru, J. A., et al., Biology of Blood and Marrow Transplantation, 2:3 (1996)). This purified HSC cell dose is 100 times more than the cell dose of unmodified marrow which readily engrafts in ablated recipients. Taken together these data suggested that an additional cell in marrow, separate from HSC, facilitates engraftment of HSC in allogeneic recipients.

In a nonoverlapping proposal using normal mice, we examined which accessory cells in bone marrow were examined to determine which are critical to engraftment of HSC in allogeneic recipients using cell sorting. The addition of as few as 30,000 FC to 10,000 HSC restores engraftment of HSC in allogeneic recipients without causing GVHD. The FC is not an HSC since it alone does not rescue ablated recipients from irradiation-induced aplasia (---). Note that the administration of highly purified HSC in fully ablated MHC disparate recipients allows prolonged survival significantly beyond that for recipients of FC (▪-▪) (FIG. 3). However, virtually all animals expire within 180 days due to marrow aplasia and late graft failure. In the mouse, committed progenitor cells (that are no longer self-renewing HSC) survive and function for up to 180 days (Ildstad, S. T., et al., Transplantation Science, 3:123 (1993)). Recipients of FC plus HSC exhibit durable engraftment and survival beyond 180 days. The prolonged survival observed in recipients of HSC alone is believed to be due to the function of committed progenitors and that FC are critical to maintenance of HSC pluripotency and self-renewal. FC most likely exerts an immunoregulatory influence on HSC in the microenvironment to maintain self-renewal.

Although the concept that graft “facilitating cells,” distinct from HSC, was at first controversial, a number of groups have now confirmed their existence. Their publications have occurred in high impact journals, including Immunity and Nature Medicine. The term facilitating cell has been incorporated into the stem cell biology vernacular and two recent editorials in Nature Medicine have commented on its potential for impact clinically (Martin, P. J., et al., Nat. Med., 6:18 (2000); Li, X. C., et al., Nat. Med, 6:866 (2000)).

The Examples below demonstrate that the facilitating cell effect titrates out at 10,000 cells. With a frequency of approximately 0.5% of total cells in marrow, the fact that approximately 5,000,000 unmodified bone marrow cells are required for HSC engraftment in allogeneic recipients would predict this observation since 10,000 FC would be present in the unmodified marrow inoculum. Gandy and Weissman (Stanford) describe two populations of FC:CD8⁺/TCR⁺ and CD8⁺/TCR⁻. They conclude that the two populations combined provide the most robust facilitating effect, but that CD8⁺/TCR⁻ FC are more potent than CD8⁺/TCR⁺ FC for facilitating engraftment of allogeneic HSC. Moreover, CD8⁺/TCR⁻ have the advantage that they are NOT GVHD-producing cells, while CD8⁺/TCR⁺ FC are. The authors compared CD8⁺/TCR⁻ FC to what they referred to as CD8^{total} marrow cells (CD8⁺/TCR⁺ and TCR⁻ cells). While >10,000 CD8⁺/TCR⁻ FC were critical to HSC engraftment in allogeneic recipients, only 15,000 CD8^{total} cells were required. It would appear that two separate mechanisms explain this observation and that these data are not contradictory to that presented herein. CD8⁺/TCR⁻ FC are TROPHIC for HSC survival while CD8⁺/TCR⁺ FC promote engraftment in their partially conditioned recipients in the presence of FC (VETO).

CD8⁺/TCR⁻ FC are critical to HSC survival, while CD8⁺/TCR⁺ FC are supportive. The two function by totally different mechanisms. Conventional T-cells (CD8⁺/TCR⁺) serve as “space-makers” or effector cells to enhance engraftment. In the presence of FC, conventional T-cells enhance engraftment. They also allow engraftment to occur at a lower level of partial conditioning. However, without FC, conventional T-cells alone do not allow durable engraftment and self-renewal of the pluripotent HSC. Hence, CD8⁺/TCR⁻ FC are critical, while CD8⁺/TCR⁺ FC are supplemental to engraftment. This proposed mechanism resolves the apparent contradictory data surrounding CD8⁺TCR+ lymph node FC, γδ-TCR⁺ FC, and CD8⁺/TCR⁻ FC recently reviewed in Nature Medicine (Martin, P. J., et al., Nat Med, 6:18 (2000)).

When the establishment of chimerism in NOD recipients was attempted, four surprising and very important observations that relate directly to the present invention emerged from our prior studies: (1) NOD mice are very prone to graft failure if the donor marrow is T cell depleted. However, the approach we utilized for T cell depletion (anti-Thy1.2 plus complement or Rabbit-Anti-Mouse Brain [RAMB]) would also have depleted graft-facilitating cells. Durable engraftment of HSC in NOD mice is critically dependent upon facilitating cells, a novel CD8⁺/TCR⁻/CD3ε⁺ population in bone marrow that enhances engraftment of highly purified HSC in allogeneic recipients. (2) Unlike normal mice, where the proportion of donor:host chimerism remains stable throughout the life of the animal, mixed NOD chimeras do not remain mixed: the donor marrow exhibits a competitive advantage in that by six months the majority of mice are >90% donor. This cannot be due to a graft versus host type of mechanism since when the donor marrow is depleted of CD4+ plus CD8^{bight} cells (T cells) a similar outcome occurs. The initial percentage donor chimerism in these recipients is lower than that for recipients of unmodified marrow. However, by 12 months the percentage donor chimerism is >90%. (3) NOD mice exhibit a relative radioresistance and require more conditioning to establish durable mixed chimerism. With TBI alone, 750 cGy is required (versus 650 cGy in normal mice); if cyclophosphamide is added on day +2 the animals initially engraft but exhibit late (two to three months) graft failure; anti-CD8 mAb conditioning of the recipient reduces the TBI down to 700 cGy, but four times more mAb is required compared to normal mice; while 750 TBI is fully ablative in B 10 mice, it is not in NOD mice, since mice that are conditioned but not transplanted develop endogenous hematopoiesis and do not expire from radiation-induced aplasia. Taken together these observations suggest that the NOD marrow microenvironment is abnormal in a number of respects, including a relative paucity of FC (see preliminary results). FC are therefore even more important to establishing chimerism in NOD mice because of the abnormalities in the hematopoietic microenvironment and that removal of FC during T cell depletion is the underlying mechanism responsible for the TCD graft failure we observe in NOD recipients.

### Define the Role of FC in Establishing Mixed Chimerism and Tolerance in NOD Recipients.

The role of CD8⁺/TCR⁺ lymph node lymphocytes as FC. CD8⁺/TCR⁺ lymph node lymphocytes have also been demonstrated to facilitate engraftment in normal mice. Martin reported that T cell depleted marrow did not engraft in MHC-disparate recipients conditioned with 800 cGy TBI unless CD8⁺/TCR⁺ lymph node lymphocytes were added to the T cell depleted marrow. These data are complementary to the data of the present invention since mAb plus complement mediated cytolysis with anti-CD4 and anti-CD8 mAb would not remove all FC but would remove conventional T cells due to difference in antigenic expression. Again, the critical experiment in which CD8⁺ lymph node lymphocytes were administered with highly purified HSC was not performed. The requirement for CD8⁺ T cells to allow engraftment after 800 cGy TBI is most likely due to the need for space-making after somewhat incomplete ablation. Again it appears that CD8⁺/TCR⁻ FC are critical to maintaining HSC self-renewal and pluripotency in NOD mice while CD8⁺/TCR⁺ T cells are only supplemental to augment chimerism by vetoing alloreactive host cells. Refining the role of each cell type in engraftment in NOD mice will lead to the development of strategies to establish chimerism with a minimally toxic approach. By defining the optimal composition of the donor marrow graft we will establish chimerism in NOD recipients with a minimally toxic approach.

It appears that host T cells, NK cells, and T/NK cells in NOD mice are the primary effector cells in resistance to engraftment of donor marrow. Which NK subfamilies are present in NOD mouse marrow was evaluated and it was observed that NOD mice lack NK1.1⁺, and the 5E6⁺ and 2B4⁺ subsets of NK cells (FIG. 4). Only DX5 and ASGM1⁺ NK cells are present. When NOD recipients were pre-conditioned in vivo with anti-DX5 mAb, the minimum dose of TBI (750 cGy) that is sufficient for engraftment could not be reduced. Anti-Thy1.2 preconditioning of the recipient also did not influence the TBI. In striking contrast, pre-conditioning of NOD recipients with anti-CD4 plus anti-CD8 mAb allowed the TBI dose to be reduced to 600 cGy. It is important to note that four times more antibody is required to mediate an effect in NOD recipients compared to normal recipients. Moreover, unlike normal controls, the host target cell population is not completely removed and cells coated with mAb remain.

Treatment of NOD recipients with mAb specific for co-stimulatory molecules also reduces the minimum TBI dose sufficient for engraftment. Pretreatment of NOD recipients with anti-CD40L (CD 154) or anti-CTLA-4 (CD 152) mAb reduced the TBI dose significantly (FIG. 5). Interestingly, when the two mAb were combined, the effect was not potentiated. In continuing studies the anti-CD8 pretreatment has been combined with the anti-CD40L. While with anti-CD8 pretreatment alone 700 cGy TBI is required for durable engraftment, the addition of anti-CD40L reduces the TBI to 600 or even lower. Experiments are in progress to determine how low this combination of mAb will allow the radiation dose to be reduced and to combine the most effective antibodies to determine whether the TBI can be eliminated completely.

Host factors that influence engraftment: Strategies to Partially condition to establish mixed chimerism. Until recently it was hypothesized that fully ablative conditioning was required for engraftment of allogeneic marrow. Conditioning consists of two components: immunosuppression to prevent immediate destruction of the newly infused marrow and cytoreduction or myelosuppression to prepare vacant niches in the hematopoietic microenvironment (Santos, G. W., et al., N. Engl. J. Med., 309:1347 (1983); (Parkman, R., et al., Clinical Immunology and Immunopathology, 40:142 (1986); Speck, B., et al., Biomedicine and Pharmacotherapy, 37:139 (1983); Papa, G., et al., Leukemia Research, 10:1469 (1986)). Both types of agents are usually required for durable engraftment even with full ablation. Most conditioning approaches include a combination of cytoreductive (myeloablative) agents such as irradiation or busulfan with immunosuppressive agents (i.e. cyclosporine, FK506, and fludarabine mycophenolate mofetil). In strategies for partial conditioning, the immunosuppressive component of conditioning becomes the most important feature. With the proper choice of immunosuppressive agents, or with effective elimination of the critical host effector cells that mediate alloresistance, engraftment can be achieved with ≦200 cGy TBI (Colson, Y. L., et al., J. Immunology, 157:2820 (1996); Storb, R., et al., Blood, 89:3048 (1997)).

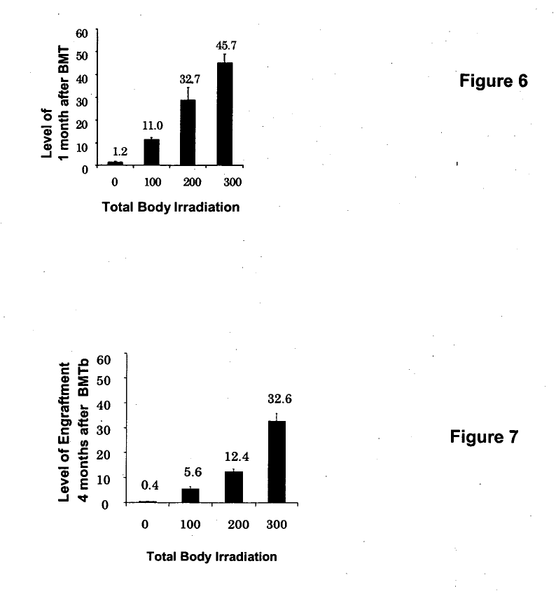

Mixed chimerism can be established with only partially ablative conditioning if the appropriate combination of immunosuppressive plus myelosuppressive agents is utilized. In normal mice, 650 cGy of TBI is required for durable engraftment of MHC-disparate marrow. The addition of 200 mg/kg cyclophosphamide on day +2 following the TBI and marrow infusion reduces the minimum TBI dose to 550 cGy (Colson, Y. L., et al., J. Immunology, 157:2820 (1996)). The addition of anti-CD8 mAb (1 mg on day-3) further reduces the TBI dose to 200 cGy (Colson, Y. L., et al., J. Immunology, 157:2820 (1996)). Doubling the marrow dose from 15×10⁶ cells to 30×10⁶ reduces the TBI dose to 100 cGy in combination with cyclophosphamide (day +2) and anti-CD8 mAb conditioning. Finally, mice defective in production of CD8⁺ cells (CD8 knockout [KO] mice) engraft without TBI as long as cyclophosphamide is administered on day +2 relative to BMT. Therefore, a cyclophosphamide-sensitive CD8⁺ cell in marrow is one host cell type responsible for alloresistance to marrow engraftment in normal recipients. The level of chimerism in these recipients is correlated with the TBI dose (FIGS. 6 and 7). All recipients are tolerant to donor in vivo and in vitro.

The mechanism of action of the cyclophosphamide is believed to eliminate alloreactive T cells in the recipient that are becoming activated to reject the donor marrow. One observation critical to this proposal is the fact that while cyclophosphamide is effective on day +2 relative to the TBI and marrow infusion, it does not work if administered on day −2, −1, 0, or +1. Moreover, mice lacking production of conventional αβ and γδ-T cells (TCR βδ KO mice) as recipients do not require cyclophosphamide for engraftment, but do require a low dose (>50 cGy) of TBI to establish durable chimerism and tolerance. Therefore, the critical mechanism for alloresistance to engraftment in normal mice is via host αβ-TCR⁺ and γδ-TCR⁺ T-cells which function in a non-redundant fashion. A similar role for host αβ and γβ-T cells will be operational in NOD mice, and probably in exaggerated form in light of the chronic activation state of NOD T cells, and the relative resistance of NOD T cells to undergo apoptosis.

When a partial conditioning approach according to the present invention is applied to NOD mice, a number of surprising and unexpected observations emerged: (1) NOD mice exhibit a relative radioresistance and require more conditioning for engraftment compared with normal mice; (2) NOD mice are highly sensitive to graft failure if the marrow is T cell depleted prior to transplantation; and (3) higher numbers of cells are required for engraftment in NOD mice compared to normal recipients (summarized in detail in preliminary results and prior work section). An understanding of the mechanism underlying these abnormalities is critical to establish a minimal conditioning approach to mixed chimerism in NOD mice.

Higher doses of unmodified marrow are required to radioprotect NOD mice.

Full ablation allows one to evaluate the influence of the composition of the marrow on engraftment yet minimize the influence of residual host cells on engraftment. In this case, most host-anti-donor alloreactive host cells have been removed. When normal mice are fully conditioned, as few as 5×10⁶ allogeneic BMC are sufficient to radioprotect 100% (Kaufman, C. L., et al., J. Immunology, 158:2435 (1997)). In striking contrast, a minimum of 30×10⁶ allogeneic BMC are required to radioprotect NOD recipients. Moreover, unlike disease-resistant controls, only 92% of recipients engraft even at that higher cell dose.

The composition of the donor marrow inoculum influences chimerism in NOD mice. When the donor marrow was depleted of CD4⁺ plus CD8⁺ cells, more cells (45×10⁶ versus 30×10⁶) were required to achieve durable engraftment in NOD recipients compared with unmodified BMC (Li, H., et al., J Immunology, 156:380 (1996)) for recipients of 5×16TCD NOD marrow plus CD4 plus CD8 depleted allogeneic marrow. The level of chimerism that resulted was also significantly lower (58±34 versus 94±6) at four weeks. However, within 12 months the percentage of donor chimerism in those animals had increased to ≧90%. The mechanism for the competitive advantage of normal marrow versus endogenous NOD marrow has not been defined, but it is unlikely to be due to a donor versus host GVH response since it also occurs when donor marrow is TCD.

In additional studies marrow of CD4⁺, CD8^{bright}, αβ-TCR⁺, or T cells plus FC together was depleted. NOD recipients were conditioned with 950 cGy TBI, a dose of irradiation is not fully ablative in NOD recipients. All mice that received unmodified marrow engrafted irrespective of bone marrow cell dose (15×10⁶, 30×10⁶, 45×10⁶). Removal of CD4⁺ or CD8^{bright} cells from the marrow did not impair engraftment. However, considerably lower levels of donor chimerism resulted when the CD8^{bright} cells were depleted from the donor marrow. No donor engraftment occurred when αβ-TCR⁺ or αβ-TCR⁺ plus FC were removed. While depletion studies suggest a trend, sorting is more informative and overcomes a number of the limitations associated with complement-mediated lysis or ferromagnetic bead depletion: (1) purity issues; (2) heterogeneity of composition; (3) incomplete removal of target cells (at best one to two logs can be removed by negative selection procedures). While one can conclude an important role for T cells in mediating engraftment in NOD recipients, one cannot exclude a critical role for FC if the marrow depleted of αβ-TCR⁺ T cells is being rejected by radioresistant NOD host cells rather than that the conventional T cells provide a TROPHIC effect for HSC survival in the microenvironment of NOD recipients. FC are critical to HSC self-renewal in NOD recipients and that αβ-T cells provide space-making capacity to neutralize the alloreactive and chronically activated NOD host cells that mediate marrow rejection.

NOD mice are relatively radioresistant. If B10.BR or B10 mice are conditioned with >950 cGy of TBI, 100% mortality results following BMT. IF BMT is not performed, the majority of conditioned animals expire with >700 cGy TBI. In striking contrast, when NOD mice are conditioned with 1000 or 1050 cGy of TBI and transplanted, minimal mortality results. Therefore, a relative radioresistance to conditioning is present in NOD mice. This finding has been independently confirmed. Other mouse models for autoimmunity have also been reported to exhibit a relative radioresistance, including the MRL mice (Ikehara, S., et al., Immunology, 86:3306 (1989)) and HLA B27 transgenic rats (Bilous, R. W., et al., N. Engl. J. Med., 321:80 (1989)). Moreover, while 100% of NOD recipients conditioned with 750 cGy TBI resume endogenous hematopoiesis if no BMT is performed, only 5% and 50% of B10.BR and B10 mice do respectively (Li, H., et al., J. Immunology, 156:380 (1996)). As a result of these studies, 1000 cGy TBI is now utilized as our fully ablative conditioning dose for NOD recipients. Secondly, by defining the mechanism responsible for this relative resistance to conditioning and how chimerism overcomes the autoimmune process a better understanding of the mechanism of disease will result. The defect in apoptotic machinery influences the autoimmunity as well as contributing to the radioresistance.

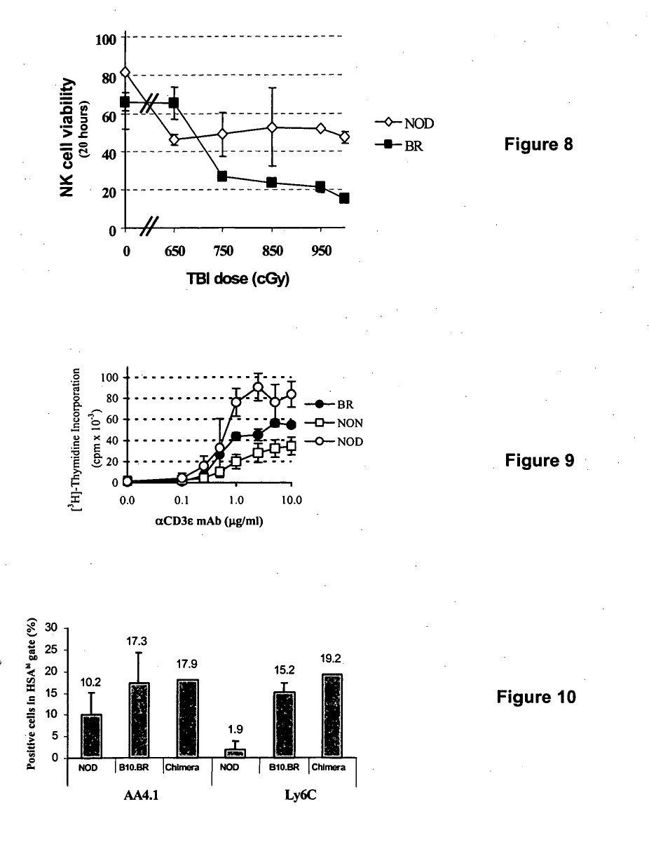

Bone marrow cells from NOD mice are relatively resistant to apoptosis induced by irradiation in vitro. In a preliminary study, bone marrow cells from both NOD and B10.BR mice were subjected to increasing does of irradiation (650-1000 cGy). At various times after irradiation, subsets of the bone marrow were analyzed for viability via flow cytometry. Results from this study indicate that NK cells from NOD bone marrow are more resistant to these irradiation doses than those from B 10.BR mice (FIG. 8). Specifically, 40-50-% of the NK cells from NOD mice exposed to 650-1000 cGy of radiation remained viable in culture for 20 hours (p<0.05). Although the B10.BR NK cells that received a radiation dose of 650 cGy exhibited similar viability to those of similarly treated NOD cells, the viability decreased to 15% when the irradiation dose was increased to 1000 cGy. In contrast, FC and HSC populations of irradiated NOD and B10.BR bone marrow cells did not exhibit significant differences. These data indicate a specific cell population (NK cells) in NOD mice is significantly more resistant to the effects of radiation when compared to a diabetes-resistant strain of mice. Additional studies are in progress to compare other cell populations and other strain combinations.

Alternate methods for conditioning the recipient. Mice of different strains provide a reasonable model to study the role of MHC loci on engraftment or graft failure due to different MHC loci and genetic backgrounds (Kaufman, C. L., et al., Blood, 84:2436-2446 (1994); Lechler, R., et al., Curr. Opin. Immunol., 3:715-721 (1991); Lowin-Kropf, B., et al., J. Immunol., 165:91-95 (2000); and Meyer, D., et al., Immunobiology, 197:494-504 (1997)).

The mouse strain combinations tested included MHC-match, minor histocompatibility, major plus minor histocompatibility mismatches, MHC-class I or class II disparate and MHC class I or class II deficient. The strain combinations were chosen so that donor and recipient hematopoietic cell contribution could be distinguished at the MHC locus. HSC are defined by the following combination of cell surface markers:Sca-1⁺/C-kit⁺/Lin⁻. Cells with this phenotype have been found to contain a population of cells with long-term multilineage reconstitution potential. (Allcock, R. J., et al., Immunol. Today, 21:328-332 (2000); Bix, M., et al., Nature, 349:329-331 (1991); Ohlen, C., et al., Eur. J. Immunol., 25:1286-1291 (1995); Schuchert, M. J., et al., Nat. Med., 6:904-909 (2000); Shenoy, S., et al., Clin. Exp. Immunol., 112:188-195 (1998); and Shizuru, J. A., et al., Biol. Blood Marrow Transplant., 2:3-14 (1996)). The data discussed in detail below, demonstrate that the purified HSC engraft readily in MHC-match (BR→BR) or minor antigen disparate recipients (BR→AKR), but not in fully MHC-disparate recipients (BR→B10). Highly purified HSC in MHC-disparate recipients allow prolonged survival. However, all animals expire within 180 days due to marrow aplasia and late graft failure. These results suggest that committed progenitor cells (that are no longer self-renewing HSC) survival and function for up to 180 days (Ildstad S.T., Transplantation Science, 3:123 (1993)).

Previous studies have indicated that B6 β2m-/- (class I deficient) mice marrow did not engraft in MHC-matched (C57BL/6×129) F₂ normal mice after lethal radiation of recipients, suggesting that rejection of class I-deficient cells is mediated by normal NK cells (Domen, J., et al., J. Exp. Med., 191:253-264 (2000); Grigoriadou, K., et al., Eur. J. Immunol., 29:3683-3690 (1999); Lowin-Kropf, B., et al., J. Immunol., 165:91-95 (2000); Spangrude, G. J., et al., Science, 241:58-62 (1998); Spangrude, G. J., et al., Blood, 78:1395-1402, (1991); Stoltze, L., et al., Today, 21:317-319 (2000); Uchida, N., et al., J. Clin. Invest., 101:961-966 (1998); Ugolini, S., et al., Curr. Opin. Immunol., 12:295-300 (2000); and Vallera, D. A., et al., Transplantation, 57:249-256 (1994)). All donor B6 β2m (class I deficient) HSC failed to engraft in B6 mice, while all Abb (class II deficient) HSC engrafted in B6 mice, strongly suggesting that the molecules of MHC class I contribute to engraftment.

To determine which MHC molecule is required for HSC engraftment, mice matching at certain MHC loci but disparate at other loci were tested. Inbred mouse strain combinations congenic for all except specific MHC class I and class II loci were utilized as recipients. Again, the data discussed in detail below demonstrate that MHC class I D is not essential for HSC engraftment since 100% animals engrafted in B10.BR→B10.A (2R) combinations and survival over 180 days. However, if the MHC-disparate at class I K locus in B10.BR→B10.MBR combinations, 17% animals engrafted of HSC and survival over 180 days. Therefore, class I K is important to HSC engraftment. Furthermore, in mice transplanted across the MHC-disparate class I K and class II I-A loci (B10.BR→B10.A(5R)), animals show poor engraftment of HSC, about 25% animal survival over 180 days. Further, indicating importance of class I K and possibly class II IA in HSC engraftment. In striking contrast, if the donor and recipient are matched at class I K and class II IA in B10.BR→B10.A (4R), 83% animals show long-term survival over 180 days and exhibited durable mixed chimerism of all the lymphoid (T and B lymphocytes), NK, and myeloid (macrophages, granulocytes) cell populations. Moreover, chimeras exhibited donor-specific tolerance in vitro.

In a previous study, it was shown that FC (CD8⁺/TCR⁻) promotes allogeneic HSC engraftment across major and minor histocompatibility complex barriers without causing GVHD. When the addition of FC plus HSC was administered to allogeneic recipients, successful engraftment resulted, and animals exhibited stable multilineage chimerism and donor-specific transplantation tolerance (Bix, M., et al., Nature, 349:329-331 (1991)). Our data showed that by transplanting 5000 purified HSC plus 30,000 FC from donor B10.BR mice into lethally irradiated, MHC-disparate allogeneic B10.A (5R), B10.MBR, C57BL/10 and B10.A (4R) recipients, 100% of the animals engrafted and exhibited long-term survival with durable mixed chimerism. These results strongly demonstrated that the FC is critical for engraftment of HSC in MHC-disparate recipients.

The mechanism of FC (CD8⁺/TCR⁻) population enhances engraftment of allogeneic HSC may be related to that of HSC expression at the MHC loci. It is hypothesized that the FC influences survival of HSC by direct interaction. Consequently, it was further determined which MHC locus requires recognition of FC. Our data showed that 100% of the animals engrafted if there were HSC and FC matching at MHC class I K locus. In contrast, 50% to 62% of the animals engrafted if there was HSC and FC mismatching at H-2 or MHC I K. These data suggest that receptor-MHC Ligand interaction plays a dominant effect.

The data indicate that recipient and donor matching at the class I D is not essential for HSC engraftment. Moreover, matching at MHC class II I-E is not essential for HSC engraftment when I-E is not expressed. In striking contrast, MHC disparate at the class I K locus results in significantly impaired engraftment of HSC. The addition of as few as 30,000 facilitating cells (CD8⁺/TCR⁻) can restore engraftment of HSC in allogeneic recipients without causing GVHD. Further, if facilitating cells and HSC match at the MHC class I K, facilitating cells have a strong biologic effect on engraftment in allogeneic recipients. These results demonstrate that MHC class I K is an essential molecule for engraftment of allogeneic HSC. The method of this invention comprises achieving a higher rate of allogeneic hematopoietic stem cell engraftment by either (i) matching the major histocompatibility complex class I K locus between donors and recipients or (ii) identifying how class I K on HSC interact with FC (CD8/33 Kd receptor complex) works thus allowing one to bypass the need for FC.

Alternatively, the recipient may be conditioned using anti-sense DNA technology, non-lethal doses of irradiation, cell type-specific antibodies, cell-type specific cytotoxic drugs or a combination thereof. In particular, the present invention encompasses an approach to make space in a recipient's bone marrow by targeting only critical cell populations in the hematopoietic microenvironment.

The invention is discussed in more detail in the subsections below, solely for the purpose of description and not by way of limitation. For clarity of discussion, the specific procedures and methods described herein are exemplified using a murine model; they are merely illustrative for the practice of the invention. Analogous procedures and techniques are equally applicable to all mammalian species, including human subjects.

The present invention culminates from the initial evaluation and identification of which specific cell populations in the host hematopoietic microenvironment are the gatekeepers for engraftment of allogeneic marrow using knockout mice (KO). In these animals (KO mice) the gene for the expression of certain cell surface molecules is disrupted so that they cannot produce these cells. Thus, no residual cells are present in these animals. Typically, a minimum of 700 cGy of TBI is required for conditioning in normal mice. In striking contrast, however according to the present invention, durable multi-lineage engraftment of allogeneic marrow was achieved with only 300 cGy TBI in mice lacking both αβ and γδ cells (TCR-β/δ KO) suggesting that host αβ-TCR and γδ-TCR cells play a critical role in allorejection. In order to characterize the minimum effective TBI dose that allows allogeneic engraftment in TCR-βδ KO mice, recipients (H-2b, that are deficient in producing functional αβ- and γβ-TCR T-cells) were conditioned with 0 to 300 cGy TBI and transplanted with 15×106 B10.BR (H-2k, having genes for the production of β-chain and δ-chain of TCR disrupted) bone marrow cells. Chimerism was assessed by flow cytometric analysis. 100% of mice conditioned with 300, 200 or 100 cGy TBI engrafted and the levels of donor chimerism were 76.1+10.4%, 52.4+30.4% and 13.5+14.3%, respectively. 85.7% of TCR β/δ double KO mice engrafted without any TBI conditioning with 1.5+0.51% of donor chimerism on 28 days. The engraftment was durable as assessed monthly for up to 6 months. The level of chimerism for all groups was directly correlated with the degree of conditioning.

Donor-type skin grafts were accepted by chimeras, while the third-party NOD (H2 Kd) skin grafts were rejected promptly. The results of this study suggest that durable chimerism and donor-specific tolerance could be achieved in mice deficient in producing functional αβ-TCR and γδ-TCR cells even without any conditioning. Targeting αβ-TCR+ and γδ-TCR+in the recipient hematopoietic environment could provide a valuable strategy in the development of clinical protocols for induction of mixed allogeneic chimerism resulting in donor-specific tolerance with minimum morbidity.

Methods for targeting αβ-TCR+ and γδ-TCR+in the recipient hematopoietic environment is discussed in further detail below and therefore the present invention encompasses and contemplates the use of antibodies, antisense DNA technology and non-lethal doses of irradiation as ways of depleting and preferably eliminating αβ-TCR+ and γδ-TCR+ cells in the recipient hematopoietic environment.

Various procedures known in the art may be used for the production of polyclonal antibodies to antigens of cells making up the hematopoietic microenvironment of the host, including but is not limited to αβ-TCR+, γδ-TCR+, and/or CD8+ cells. For the production of antibodies, various host animals can be immunized by injection with purified or partially purified hematopoietic cells such as stromal cells including but not limited to rabbits, hamsters, mice, rats, etc. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, Ricin and potentially useful human adjuvants such as BCG (bacille Calmette-Guerin) and Corynebacterium parvum.

A monoclonal antibody to antigens of αβ-TCR+, γβ-TCR+, and/or CD8⁺ cells may be prepared by using any technique which provides for the production of antibody molecules by continuous cell lines in culture. These include but are not limited to the hybridoma technique originally described by Kohler and Milstein, Nature, 256:495-497 (1975), and the more recent human B-cell hybridoma technique (Kosbor, et al., Immunology Today, 4:72 (1983); Cote, et al., Proc. Natl. Acad. Sci., USA, 80:2026-2030 (1983) and the EBV-hybridoma technique (Cole, et al., Monoclonal Antibodies and Cancer Therapy, Alan R. Liss, Inc., pp. 77-96 (1985)). Techniques developed for the production of “chimeric antibodies” by splicing the genes from a mouse antibody molecule of appropriate antigen specificity together with genes from a human antibody molecule can be used (e.g., Morrison, et al., Proc. Natl. Acad. Sci. USA, 81:6851-6855 (1984); Neuberger, et al., Nature, 312:604-608 (1984); Takeda, et al., Nature, 314:452-454 (1985). Such chimeric antibodies are particularly useful for in vivo administration into human patients to reduce the development of host anti-mouse response. In addition, techniques described for the production of single chain antibodies (U.S. Pat. No. 4,946,778, which is incorporated herein by reference) can also be adapted.

Such antibody conjugates may be administered to a human patient prior to or simultaneously with donor cell engraftment. It is preferred that these conjugates are administered intravenously. Although the effective dosage for each antibody must be titrated individually, most antibodies may be used in the dose range of 0.1 mg/kg-20 mg/kg body weight. In cases where sub-lethal doses of irradiation are used, total body irradiation (TLI) of a human recipient may be administered up to 7.5 Gy as a single dose or a combined total of 22 Gy administered in fractionated doses. Alternatively, TBI may be administered up to about 5.5 Gy.

The use of antisense strategies presents a theoretically simple tool to identify, with exquisite precision, the molecular mechanisms responsible for various cellular processes. It is based on the fact that each protein synthesized by a cell is encoded by a specific messenger mRNA (mRNA). If translation of a specific RNA is inhibited, the protein product derived from this translation will likewise be reduced. Oligonucleotide sequences, can therefore be designed to be complementary (antisense) to a specific target mRNA sequence, such as the β-chain and/or the δ-chain of TCR, and because of this complementarity, it will bind to the target sequence thereby inhibiting translation of that specific mRNA. An antisense oligonucleotide complementary to a particular mRNA is referred to herein as being “directed against” the product of translation of that message. It is believed that an antisense oligonucleotide, by hybridizing to the RNA and forming a complex, blocks target mRNA ribosomal binding causing translational inhibition. Alternatively, the duplex that is formed by the sense and antisense molecules may be easier to degrade. Other theories describe complexes that antisense RNA could form with complementary DNA to inhibit mRNA transcription. Thus, an antisense oligonucleotide might inhibit the translation of a given gene product by either directly inhibiting translation or through inhibition of transcription.

Apoptotic cell death is an important mechanism of maintaining homeostasis in the immune system and for regulating the fate of lymphocytes following encounter with self- and foreign antigens. Nonfunctional T cells as well as autoreactive T cells are eliminated by apoptosis. The best characterized regulators of apoptosis in T cells are the members of the Fas and Bcl families. Fas (CD95) induces apoptosis in mature, activated T cells when they are repeatedly stimulated by antigen and maintains T cell tolerance by deleting autoreactive cells (Van Parijs, L., et al., Curr. Opin. Immunol., 8:355 (1996)). The Bcl family proteins, especially Bcl-2 and Bcl-x, prevent T cells from undergoing apoptosis after growth-factor withdrawal and after exposure to irradiation (Yin, X. M., et al., Cold Spring Harb. Symp. Quant. Biol., 59:387-93:387 (1994); Cory, S., et al., Annu. Rev. Immunol., 13:513 (1995)). Whether these pathways serve distinct or overlapping functions is an issue of considerable uncertainty. Some experiments with transfected lymphoid cell lines and studies of hepatocytes in Bcl-2 transgenic mice have shown that Bcl-2 can inhibit Fas-mediated apoptosis (Itoh, N., et al., J. Immunol., 151:621 (1993); Rodriguez, I., et al., J. Exp. Med., 183:1031 (1996); Armstrong, R. C., et al., J. Biol. Chem., 271:16850 (1996)). Other experiments have shown that Bcl-2 does not block apoptosis induced by the Fas pathway (Van Parijs, L., et al., Curr. Opin. Immunol., 8:355 (1996)). It is likely that the effects of Bcl-2 depend upon level of expression or vary with cell types. The function role of Fas and Bcl proteins has not been fully evaluated in the context of reversal of the autoimmune process by mixed chimerism in NOD mice.

Host CD8⁺/NK cells and αβ plus HOST γδ-T cells each contribute to alloresistance to engraftment in normal mice. At least three main cell types contribute to alloresistance to engraftment. Knockout (KO) mice deficient in production of specific cell types have proven invaluable in these studies. In CD8 KO mice, durable engraftment and tolerance can be achieved without irradiation as long as cyclophosphamide is administered on day +2 relative to marrow infusion. In TCR β KO mice which produce γβ-TCR⁺ T cells but not αβ-T cells, engraftment is only achieved if the mice are conditioned with 300 cGy TBI plus cyclophosphamide on day +2. If the cyclophosphamide is omitted, no donor engraftment occurs. In TCR β KO mice, which produce αβ-TCR⁺ T cells but not γβ-T cells, only 56% engraft after conditioning with 300 cGy TBI plus cyclophosphamide. These data confirm that in normal mice, αβ-T cells, and to a somewhat lesser extent γβ-T cells, play a major role as effector cells in host marrow graft rejection (manuscript submitted).

In TCR βδ-KO mice in which no αβ or γδ-T cells are produced, engraftment occurs without cyclophosphamide or irradiation. However, if minimal TBI (i.e., ≧50 cGy) is not administered, only very low (˜1%) levels of chimerism result. The level of chimerism is directly correlated with the dose of TBI. The profile of T/NK and NK subfamilies in the marrow differs significantly between the CD8 KO and βδ KO mouse strains. In TCR βδ KO mice, T/NK cells are absent, while in CD8 KO mice the CD8⁺/NK⁺/CD3ε⁺ subpopulation is absent. Taken together, these data indicate that conventional T cells as well as T/NK cells in normal mice play a significant role in resistance to engraftment of allogeneic marrow. In light of the fact that T cells from NOD mice are relatively resistant to apoptosis and in a chronically activated state, it is possible that this in part contributes to the relative resistance to engraftment and conditioning in NOD recipients. By defining those specific donor and host factors that influence engraftment in NOD mice we will identify strategies to apply mixed chimerism to induce tolerance with minimal toxicity.

Peripheral T cells in NOD mice are in a chronically more activated state compared to normal mice. NOD splenocytes proliferate to a higher degree and have a more activated phenotype as assessed by ³H-thymidine incorporation after culture on anti-CD3 mAb coated plates compared to splenocytes from B10.BR or NON control mice (FIG. 9). Although this chronic activation should make them more prone to undergo apoptosis, the converse is observed, especially for CD4⁺ T cells (Leijon, K., et al., Int. Immunol., 6:339 (1994); Garchon, H. J., et al., Eur. J. Immunol., 24:380 (1994)). An understanding of the mechanism of this relative resistance to apoptosis in naïve NOD mice and how mixed chimerism induces tolerance and restores normal censoring of autoreactive T cells in the chimeras will provide strategies to specifically optimize the outcome.

### Define the Mechanism by Which Chimerism Reverses Autoimmunity in NOD Mice.

The identification of the mechanism by which chimerism restores self-tolerance in NOD mice will provide a better understanding of the development of disease and may have practical implications of disrupting or augmenting these pathways.

In summary, the overall focus of the present application is to establish a clinically relevant approach to apply HSC chimerism to restore self-tolerance and induce allogeneic tolerance in type I diabetes.

The central focus of the present invention has been to develop clinically applicable strategies to induce donor-specific tolerance in NOD mice using mixed chimerism. A second benefit of equal importance that has emerged from these studies is the recognition that mixed chimerism also reverses the systemic autoimmune process in NOD recipients.

The original focus of research in diabetes was to determine whether HSC chimerism would induce tolerance to islet allografts in diabetic NOD recipients. NOD mice were monitored daily until they developed insulin-dependent diabetes. The diabetic mice were conditioned with 950 cGy TBI and transplanted with donor-specific marrow plus islet transplants. The donor-specific islets were permanently accepted, and MHC-disparate third party allografts were rejected with a time course similar to unmanipulated controls (Li, H., et al., Transplantation, 57:592 (1994)), indicating a robust, donor-specific tolerant state. There was no evidence for recurrent insulitis or chronic rejection on histologic examination of the islet tissue. When NOD mice were transplanted at 12 weeks, a time point where active autoimmunity is present but insulin-dependence has not yet developed, the development of the terminal complications of diabetes was prevented (Li, H., et al., J, 156:380 (1996)).

NOD mice are prone to graft failure. Graft versus host disease remains a major limitation in BMT (Ferrara, J. L., et al., N. Engl. J. Med.,, 324:667 (1991)). Historically, depletion of T cells from bone marrow prevented GVHD but was associated with a very high rate of failure of engraftment in humans (Blazar, B. R., et al., New York, N.Y. Liss:1987) (Vallera, D. A., et al., Transplantation, 47:751 (1989); Filipovich, A. H., et al., Transplantation, 44:62 (1987)). The NOD mouse is also highly susceptible to graft failure after TCD. When donor marrow is depleted of T cells using RAMB or anti-thyl, >92% of the recipients fail to engraft with donor marrow, even with fully ablative conditioning (Kaufman, C. L., et al., J. Immunology, 158:2435 (1997)). It appears that it is depletion of FC more than conventional T cells that is responsible for the exaggerated failure of engraftment observed in NOD recipients. The data below suggest that in the NOD recipient, the microenvironment is impaired, resulting in a stringent requirement for FC to maintain durable engraftment and self-renewal of donor HSC.

Marrow from NOD mice contains FC, but the proportion of CD3ε⁺ FC is significantly lower. The biologic activity of graft facilitation accompanies the CD3ε⁺ population in marrow. While the relative percentage of CD3ε⁺ FC are 5-10% in marrow of most normal mice (Gandy, K. L, et al., Immunity, 11:579 (1999); Kaufman, C. L., et al., Blood, 84:2436 (1994); Colson, Y. L., et al., Nature Medicine, Submitted: (1999)), only 0.7% of CD8⁺/TCR⁻ FC in NOD mice are CD3ε⁺. In normal animals, CD3E is a critical molecule to FC function since CD3 transgenic mice which do not produce the CD3ε molecule on the surface of their cells do not have FC. This observation may explain the tendency toward graft failure observed in NOD recipients following BMT and may also explain in part why the NOD/SCID mouse is such a good recipient for testing engraftment of human marrow (Shultz, L. D., et al., J. Immunol., 164:2496 (2000)).

### Characterize the Hematopoietic Defects in NOD Mice

In previous studies, a phenotypic abnormality in bone marrow from NOD mice was identified and demonstrated to be associated with a functional abnormality in CFU-C formation by myeloid progenitor cells in response to IL-3, GM-CSF, and IL-5. While disease-resistant BALB/c mice exhibit a predicted staining profile for HSA⁺/Ly6C⁺ double positive cells with four staining profiles (HSA^{hi} Ly6C⁻; HAS⁺Ly6C^{lo}; HSA⁺Ly6C^{hi} (the most abundant population); HSA^{lo} Ly6C^{hi}), bone marrow from NOD mice is aberrant (Langmuir, P. B., et al., International Immunology, 5:169 (1993)). In NOD mice, the Ly6C expression is absent in the HSA^{hi} and HSA⁺ subsets, but is still present in the HSA^{lo}population. Similarly, bone marrow from BALB/c marrow stained with HSA plus AA4.1 is predominantly HSA^{hi} AA4.1⁺ Ly5C⁻ (Langmuir, P. B., et al., International Immunology, 5:169 (1993)). In striking contrast, marrow from NOD mice contains very few HSA^{hi} AA4.1⁺ cells. Interestingly, the marrow from NOD chimeras exhibited the phenotype characteristic of normal mice, suggesting a relative competitive advantage for normal marrow over endogenous disease-prone NOD (FIG. 10) (p<0.05).

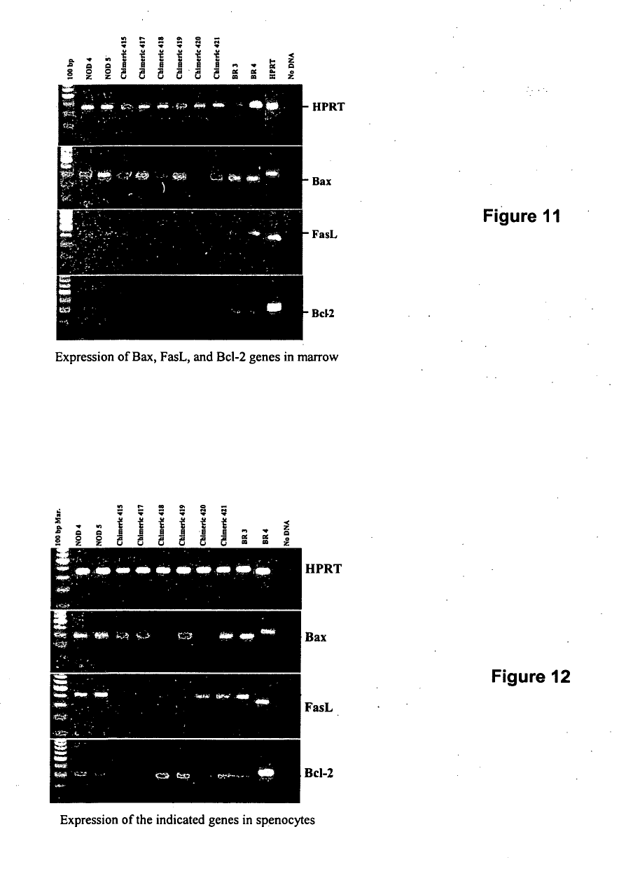

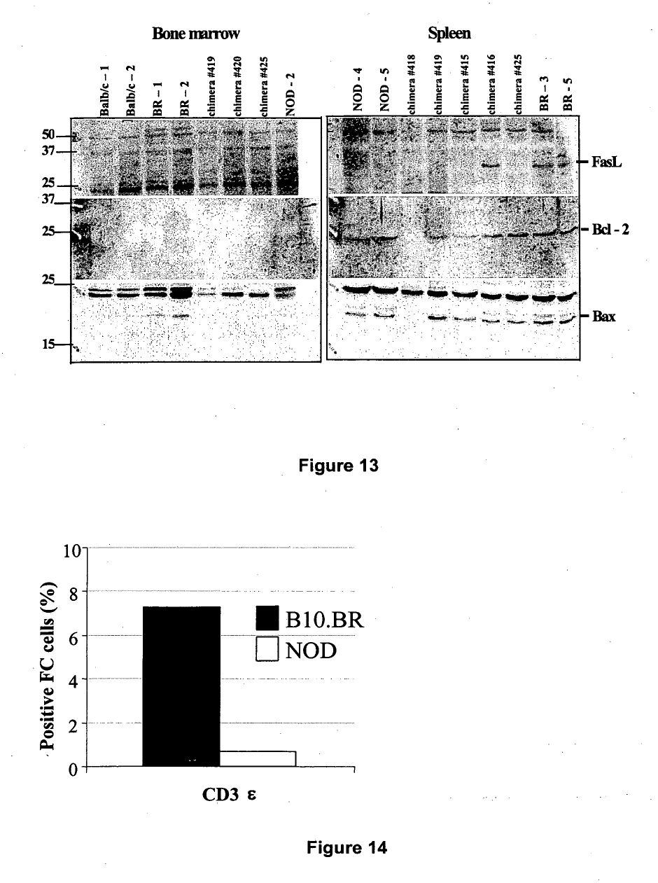

Although the molecular mechanism by which diabetes is treated with mixed chimerism is not known, it is believed that donor B 110.BR cells in chimeras restore defective apoptosis in the NOD mice that leads to elimination of autoreactive lymphocytes and abrogation of the diabetes. The expression of the death receptor and ligand (Fas and FasL), pro-apoptotic Bax and anti-apoptotic genes Bcl-2 in chimeras, compared to NOD mice and normal controls is currently being analyzed. Bone marrow cells and splenocytes were harvested from chimeras at 4, 8, and 11 weeks and age-matched NOD, B 10.BR, and BALB/c as controls. These cells were then processed for protein and RNA extraction. The expression of the genes for FasL, Bcl-2, and Bax, was analyzed using oligonucleotide primers specific for each gene in RT-PCR reaction and specific antibodies in Western blots. The expression of the housekeeping gene HPRT was used to control the quality and quantity of RNA in each sample. Equal amounts of the normalized cDNA mixture were then used for PCR amplification. PCR samples with no cDNA served as controls for PCR contamination (FIG. 11; no DNA). In general, a major difference in the expression of Bcl-2 and Bax in NOD mice compared to BALB/c and B 110.BR control was not detected. The expression of Bax in bone marrow cells as well as splenocytes of control animals and chimeras was detected. Bax was expressed at higher levels in splenocytes as compared with marrow as detected by both Western blot and RT-PCR (FIGS. 11 and 12). In contrast, Bcl-2 was only detectable in splenocytes of allogeneic chimeras and control NOD, BALB/c, and B 10.BR normal animals. FasL was expressed both in marrow and splenocytes of chimeras and control animals. Similar to Bax, the expression of FasL was higher in splenocytes as compared with marrow cells. Chimeras, however, expressed strikingly different levels of FasL at the protein level, some demonstrating normal levels of expression others demonstrating almost undetectable levels of expression (FIG. 13). There was no detectable phenotype associated with this variable level of FasL expression related to either the level of chimerism nor the state of diabetes. Although significant variations in the level of FasL, Bcl-2, and Bax molecules between diabetes resistant BALC/c and B10.BR strains versus diabetes-prone NOD were not detected, this may be because of the analysis of the whole bone marrow and splenocytes rather than isolated cell populations, such as CD4⁺ and CD8⁺ T cells. Additionally, the expression of these genes in NOD may depend on the age of the animals and the status of the autoimmune disease.

The invention is further illustrated by the following non-limiting examples. All scientific and technical terms have the meanings as understood by one with ordinary skill in the art. The specific examples which follow illustrate the methods in which the present invention may be performed and are not to be construed as limiting the invention in sphere or scope. The methods may be adapted to variation in order to be embraced by this invention but not specifically disclosed. Further, variations of the methods to produce the same compositions in somewhat different fashion will be evident to one skilled in the art.

Chimera Preparation. Chimeras will be prepared as previously described (Li, H., et al., Transplantation, 60:59 (1995)). Using sterile technique marrow from the tibias and femurs will be harvested, and a single cell suspension prepared. Bone marrow cells (BMC) will then either be processed for cell sorting or kept on ice until injection. The final BMC inoculum will be infused into the recipient via the lateral tail vein four to six hours after TBI.

Multi-parameter Live Sterile Cell Sorting for FC and HSC. Live sterile cell sorting will be performed to isolate purified FC and HSC (Li, H., et al., Transplantation, 60:59 (1995)). Directly labeled monoclonal antibodies (Pharmingen) will be added to the BMC at saturating concentrations and incubated at 4° C. for 30-45 minutes in the dark. Cells will be washed, filtered and re suspended to a final concentration of 2.5×10⁶ cells/ml. FC Isolation: BMC will be stained with anti-CD8 PE and anti-αβ and γδ TCR FITC mAb and the FACSVantage SE sorter (Becton Dickinson, Mountain View, Calif.) set to acquire only the FC population. HSC Isolation: BMC will be stained for SCA-1 (Ly6A/E-PE), C-kit (CD 117-APC), and the lineage-specific markers (lin) B220 (CD45R-FITC), CD8^{α} (FITC), MAC 1 (CD11b-FITC), GR-1 (FITC) and αβ-TCR (FITC). The Vantage sorter will be set to acquire only lin⁻/C-kit⁺/SCA-1⁺ cells.

Characterization of Chimerism by Flow Cytometric Analysis. PBL will be tested monthly to determine donor chimerism. Flow cytometry will be used to determine the percent donor PBL using antibodies against MHC class I cell surface markers (Li, H., et al., Transplantation, 60:59 (1995)) (FACSCalibur [Becton Dickinson] flow cytometer). PBLs from naïve animals (from donor and recipient strains) stained with antibodies positive and negative for their Class I markers will be used as controls.

Flow Cytometric Analysis of Intracellular Bcl-2 and Bcl-x. B and T cells will be tested for Bcl-2 and Bcl-x expression by three-step flow cytometric staining, as previously described (Roubenoff, R., et al., Arthritis and Rheumatism, 30:1187 (1987)). First, cell surface markers will be stained with antibodies specific for B220 and IgM or IgG; and for αβ TCR and CD4 or CD8. Second, cells will be permeabilized with 0.03% saponin detergent and stained for Bcl-2 (hamster monoclonal clone 3F11, Pharmingen) or Bcl-x (affinity-purified from rabbit polyclonal, Signal Transduction Laboratories) followed by staining with fluorochrome-conjugated anti-hamster or anti-rabbit antibodies. Stained and washed cells will be analyzed on a FACSCalibur flow cytometer (Becton Dickinson).

High-density Gene Microarray Analysis. Total cell RNA will be harvested from FC, B220+, CD4⁺ T cells, or CD8⁺ T cells and subjected to microarray analysis using Affymetrix technology and equipment available via the J. Graham Brown Cancer Center of the University of Louisville. RNA purification will be performed with the Qiagen Easy RNA purification kit (Qiagen, Inc.) with as many sorted cells as are needed to produce 151g total RNA (approximately 15×10⁶ cells). Multi-parameter live cell sorting will be performed as described above. Synthesis of cDNA, cRNA, and hybridization analyses will be performed according to the specifications of Affymetrix, Inc. Hybridization intensities corresponding to expression levels of individual mRNA species will be tabulated and compared using Affymetrix GeneChip Analysis software (Affymetrix, Inc.)

Histology. Tissues will be harvested and fixed in 10% neutral buffered formalin. Tissues will be stained with H&E by the Special Procedures Laboratory in the Department of Pathology at the University of Louisville School of Medicine. Immunohistochemistry for insulin will also be performed on pancreatic tissues.

Monitoring for Diabetes and Insulin Therapy. NOD mice will be monitored weekly for the presence of urine glucose. Upon testing positive on three consecutive days, a blood glucose measurement will be obtained to confirm a diagnosis of diabetes. Diabetic NOD mice will receive a daily dose of 2U recombinant human insulin (NovulinR, Novo Nordisk, Clayton, N.C.).

The Cell Sorting and Imagine Core is equipped with three Becton Dickinson 3 laser Vantage Flow Cytometers/Cell Sorters for multi-parameter live sterile sorting, two FACSCaliburs with four color capability with data analysis stations, Gammacell 1000 (blood cell irradiator), Wallac Betaplate Counter, and fluorescent microscopes including a Leica MPS with a photomicrographic system.

Molecular Biology Core. The Molecular Core is equipped with an ABI Prism 377 DNA Sequencer, a GS-700 Imaging Densitometer, a Gel Doc 1000 a Perkin Elmer PCR System, a Storm Phosphoimager, Biorad Gel Documentation system with automatic film processor and common computer areas with an image processing station, 2 Perkin Elmer 2400 Thermocyclers, one 9600 Thermocycler, a Stratagene Robocycler, LSB50 Luminescence spectrometer, Victor 1420 Multichannel counter, Pharmacia spectrophotometer, Pharmacia Chromatography unit with AKTA protein purification unit, Misonix sonic dismembrator with cup horn, Bellco Autoblot Hyb Oven and Hybshaker water bath, BioRad 583 Gel Dryer with a Savant GEP140 Gelpump, liquid nitrogen storage, Coulter ONYX (for human differential), Coulter Z2, BTX square electroporator, BioRad GenePulser, inverted and light microscopes including a Leica Fluorescent microscope, fume hoods and a full complement of centrifuges, CO₂ incubators, freezers, refrigerators, autoclave, glassware baking oven and general laboratory equipment.

The Animal Facility. All animals are housed in HEPA-filtered ventilated cage racks and handled in laminar-flow change stations using standard sterile technique.

### EXAMPLES

The examples herein are meant to exemplify the various aspects of carrying out the invention and are not intended to limit the invention in any way.

### Example 1

### Influence of Cellular Depletions on Engraftment

TABLE 1

| Cell | | | | Average % Donor |
|---|---|---|---|---|
| type depleted | # mice | Cell Dose | % Engrafted | Chimerism |
| None | 30 | 30-60 × 10⁶ | 100 | 98.5 ± 2.0 |
| CD8+^{bright} | 3 | 45 × 10⁶ | 100 | 77.8 ± 20.4 |
| | 5 | 60 × 10⁶ | 80 | 80.7 ± 4.6 |
| CD4⁺ | 2 | 45 × 10⁶ | 100 | 93.4 ± 5.2 |
| | 2 | 60 × 10⁶ | 100 | 96.7 ± 0.6 |
| αβ TCR⁺ | 2 | 45 × 10⁶ | 0 | 0 |
| Tcells + FC | 4 | 30 × 10⁶ | 0 | 0 |
| (RAMB) | | | | |

The FC is not efficiently removed by antibody plus ferromagnetic bead depletion, while CD8⁺/TCR⁺ T cells are. Engraftment was impaired in NOD recipients if αβ-TCR⁺ cells plus FC were removed. While not to be limited by a hypothesis it is believed that the T cells serve as space-making cells for this threshold level for ablation while the FC are critical to durable HSC engraftment. The experiments in Example 2 below will define the precise role of each cell type (CD8⁺/TCR⁻ FC versus CD8⁺/TCR⁺ T cells) in engraftment in NOD recipients. It is hypothesized that as conditioning increases, the role for αβ-TCR⁺ T cells will become less important but the role for FC will remain critical to durable chimerism. Moreover, by augmenting FC dose it is predicted that chimerism will be established at a lower TBI dose. As the precise contribution of each cell type to engraftment in NOD recipients is further defined, opportunities to potentiate the effect will emerge.

As discussed previously, NOD mice are relatively radioresistant (Li, H., et al., Transplantation, 57:592 (1994); Li, H., et al., Surgery, 118:192 (1995)). While conditioning with 950 cGy of TBI is sufficient to allow engraftment in normal disease-resistant mice, ≧1050 cGy TBI is required in NOD mice. Moreover, preliminary studies indicate that up to 1100 cGy is not supralethal, while for B10 and B10.BR mice it is.

One observation critical to the present invention is that mixed NOD chimeras do not remain mixed. Unlike normal animals, where once mixed chimerism is established it remains stable and durable for the lifespan of the recipient, in NOD mice the proportion of donor chimerism rises for all lineages until it is ≧95% donor by 6 months. Mixed chimerism most likely restores censoring of potentially autoimmune cells by providing normal pro-apoptotic machinery. It has further been discovered that bone marrow and spleen-derived lymphocytes from NOD mice express significantly higher levels of Fas.

Bone Marrow from NOD Mice Does Not Contain Functional FC. It hs been demonstrated that bone marrow from NOD mice does not contain functional FC. Marrow from NOD mice was first evaluated to determine whether it contains phenotypically characteristic FC (CD8⁺/TCR⁻/CD3ε⁺). While most wild type mouse strains contain 5-10% CD8⁺/TCR⁻ FC that are CD3ε⁺, NOD mice are lacking this population (FIG. 14).

To evaluate the function of FC, B10.BR mice were conditioned with 950 cGy TBI and reconstituted with a mixture of TCD NOD and TCD B 110.BR bone marrow cells in the absence and presence of purified NOD FC. There was no difference in chimerism, regardless of the administration of NOD FC (20-25%), suggesting that marrow from NOD mice may not contain functional FC. When FC from normal donors were administered, the percentage donor chimerism was >95%. To further evaluate whether NOD contain functional FC, purified HSC from NOD mice were transplanted with or without purified NOD FC. Engraftment occurred even in the absence of NOD FC. These data indicate that CD8⁺/TCR⁻ population of NOD FC does not have a subset that expresses CD3E, and does not appear to functionally facilitate engraftment of NOD HSC. The NOD HSC appears to have compensated for this limitation, since unlike HSC from wild type donors, it does not require FC to engraft in normal recipients. The absence of functional FC may explain why NOD mice are so sensitive to TCD graft failure and why wild type marrow exhibits a competitive advantage in NOD recipients.

In summary, the focus of the present invention is to define the role of graft facilitating cells in establishing mixed chimerism in NOD mice and determine the mechanism by which this chimerism reverses the systemic autoimmune process. A better understanding of the mechanism underlying the reversal of the autoimmunity will lead to clinically applicable strategies to optimize the outcome and minimize recipient morbidity.

NOD mice are exquisitely sensitive to TCD graft failure. It is hypothesized that FC are critically important to engraftment in these recipients and that removal of FC with the T cell procedure is the underlying mechanism for graft failure. The strategies utilized for TCD would also have removed FC. This hypothesis will be tested and the role of each bone marrow cell type in establishing chimerism in NOD recipients will be defined.

The primary focus of this the present invention is to disclose a minimal conditioning approach to establish mixed chimerism to induce tolerance in NOD recipients. Engraftment is multifactorial. Both donor and host factors influence engraftment as independent but complementary variables. By optimizing first the donor factors that influence outcome and then defining the recipient factors that resist engraftment, we hypothesize that we will achieve chimerism in NOD recipients with minimum morbidity. It is hypothesized that graft facilitating cells are central to the tendency to TCD graft failure in NOD recipients.

### Example 2

### Define the Role Of FC in Establishing Mixed Chimerism and Tolerance in NOD Mice

The first set of experiments will characterize the role of FC and conventional T cells in engraftment in NOD mice which are fully ablated. We hypothesize that the exaggerated tendency for graft failure observed in NOD mice associated with T cell depletion of donor marrow is due more to removal of the FC and to a lesser extent conventional T cells in fully ablated NOD mice. Although the role of FC will remain critical to stem cell survival and self-renewal for all doses of conditioning, the role for conventional T cells as space making or veto cells becomes increasingly more important with decreasing TBI doses.

Experiment 1: Determine Whether FC are Critical to HSC Engraftment in NOD Mice.

Experiment 1 will determine whether graft failure with tcd is due to removal of fc from donor marrow. In these initial experiments, eight to twelve week old female NOD mice will be conditioned with 1000 cGy TBI and transplanted with varying numbers of FC and HSC from B10.BR donors to evaluate the influence of FC dose on the HSC dose required for engraftment. Recall that NOD mice require at least 3 times more unmodified bone marrow cells for engraftment. In normal mouse recipients, the FC effect titrates out at 10,000 FC. It is, therefore, hypothesized that the FC effect will titer out at approximately 30,000 FC in NOD mice. However, more HSC may also be required. Therefore, both cell dose titrations (FC and HSC) will be tested.

Experiment 2: Is Graft Failure with T Cell Depletion Due to Removal of FC

| | | | CELLS DEPLETED | | |
|---|---|---|---|---|---|
| | | | FROM | | |
| | | | DONOR | CELL | |
| GROUP | N | DONOR | MARROW | DOSE | RECIPIENT |
| A | 6 | B10.BR | None | 30 × 10⁶ | NOD |
| B | 6 | B10.BR | None | 45 × 10⁶ | NOD |
| C | 6 | B10.BR | None | 60 × 10⁶ | NOD |
| D | 6 | B10.BR | αβ − TCR⁺ | 30 × 10⁶ | NOD |
| E | 6 | B10.BR | αβ − TCR⁺ | 45 × 10⁶ | NOD |
| F | 6 | B10.BR | αβ − TCR⁺ | 60 × 10⁶ | NOD |
| G | 6 | B10.BR | αβ + γδ TCR⁺ | 30 × 10⁶ | NOD |
| H | 6 | B10.BR | αβ + γδ TCR⁺ | 45 × 10⁶ | NOD |
| I | 6 | B10.BR | αβ + γδ TCR⁺ | 60 × 10⁶ | NOD |
| J | 6 | B10.BR | Thy 1.2⁺ | 30 × 10⁶ | NOD |
| K | 6 | B10.BR | Thy 1.2⁺ | 45 × 10⁶ | NOD |
| L | 6 | B10.BR | Thy 1.2⁺ | 60 × 10⁶ | NOD |

Twelve-week old female mice will be utilized in these studies. At this time point, active insulitis is present in 100% but the requirement for insulin has not developed. The age of the NOD recipients is not correlated with the ability to establish engraftment and tolerance. In that regard, all age groups for NOD mice as recipients behave similarly. All mice in Experiments 1 and 2 will be fully conditioned with 1000 cGy TBI in order to minimize the influence of host alloreactive cells (host versus donor) on engraftment as a variable. The first experiments will therefore characterize the role of cells in the donor marrow graft on engraftment in the presence of a relatively neutralized recipient microenvironment. Groups A-C are controls. Groups D-F and G-I should engraft. Groups J-L will not engraft since the FC is Thy 1.2⁺. They are also controls. Cellular depletions will be performed with cell sorting for these experiments. With immunomagnetic bead and complement-mediated lysis at best a two log depletion of the target population can be achieved. Therefore, while a positive result is informative, a negative result is not. Moreover, with cell sorting a very high degree of purity (>95%) for the target population can be achieved. In addition, the purity and therefore interpretation of the data is by far better with cell sorting. The adequacy of the cellular depletion will be confirmed by reanalysis.

An aliquot of marrow will be stained for FC in order to quantify the absolute number infused into the recipient by staining with CD8 PE/αβ plus γδ TCR⁺ FITC in two color analysis. Similarly, HSC(C-kit⁺/Sca-1⁺/lin⁻) will be enumerated, as well T cells (αβ plus γδ TCR⁺). Total cell dose/kg recipient body weight will be calculated for each recipient. These titrations will establish the approximate target cell numbers for the cell sorting experiments to follow.

Post-transplant evaluation and observation. Animals will be individually ear tagged prior to transplantation. Recipients will be monitored twice weekly for glucosuria. For each experiment a group of age-matched unmanipulated controls will be prepared. Sustained glucosuria (≧3 consecutive tests) will be confirmed by glucometer. Sustained glucosuria reliably predicts the development of diabetes. If the animals do not develop diabetes by 6 months, we will euthanize a representative number from each group (n=4) monthly thereafter and examine the pancreata for presence of insulitis as well as the submandibular glands for sialadenitis.

The pancreas will be scored according to the method described by Kagi et al. (Kagi, D., et al., J Immunol., 162:4598 (1999)). At least 40 randomly chosen islets will be evaluated. Insulitis is classified as: (1) 0=no insulitis; (2) 1=peri-insulitis with a weak peripheral inflammatory infiltrate that does not penetrate in islet tissue; (3) 2=moderate insulitis with an infiltrate <50% of islet area; (4) 3=an infiltrate of >50% of the islet area.

10,000 C-kit⁺/Sca-1+/lin⁻ HSC and 30,000 FC are required to radioprotect 100% of normal allogeneic recipients of HSC plus FC. If one back-calculates from the relative frequency of HSC (0.4% of total cells in marrow), the fact that 5×10⁶ unmodified BMC are required to engraft in normal allogeneic recipients, one would predict this approximate cell number requirement as a threshold in normal mice. Significantly more unmodified bone marrow cells are required in NOD mice for engraftment and that if donor marrow is depleted of T cells, NOD mice fail to engraft. By augmenting FC, engraftment can be improved and the total stem cell dose may be able to be decreased to a level similar to normal mice recipients.

Experiment 3: Are FC Critical to HSC Engraftment In NOD Mice

| | | | | FC | |
|---|---|---|---|---|---|
| GROUP | N | HSC DONOR | HSC # | DONOR | FC# |
| A | 6 | B10.BR | 10,000 | B10.BR | 30,000 |
| B | 6 | B10.BR | 20,000 | B10.B | 30,000 |
| C | 6 | B10.BR | 30,000 | B10.B | 30,000 |
| D | 6 | B10.BR | 10,000 | B10.B | 60,000 |
| E | 6 | B10.BR | 20,000 | B10.B | 60,000 |
| F | 6 | B10.BR | 30,000 | B10.B | 60,000 |
| G | 6 | B10.BR | 10,000 | B10.B | 90,000 |
| H | 6 | B10.BR | 20,000 | B10.B | 90,000 |
| I | 6 | B10.BR | 30,000 | B10.B | 90,000 |
| J | 6 | B10.BR | 10,000 | — | — |
| K | 6 | B10.BR | 20,000 | — | — |
| L | 6 | B10.BR | 30,000 | — | — |

Each experiment will be repeated three times. In this first experiment it will be determined whether augmenting FC dose will allow engraftment of lower doses of HSC and whether NOD mice are critically dependent upon HSC for engraftment. It is hypothesized that as FC dose increases (Groups D-F and G-I), fewer HSC will be required. Groups J-L are controls. They will exhibit prolonged survival but will not durably engraft. Mice will be monitored for chimerism monthly. Ten randomly selected animals per treatment group will be followed for 12 months for development of overt diabetes. Unmanipulated age matched controls will be prepared for each group. At 3, 6, 9, and 12 months four mice will be euthanized per treatment group and the pancreata will be evaluated for insulitis according to the method described by Kagi, et al. (Kagi, D., et al., J. Immunol; 162:4598 (1999)). Briefly, the pancreas tissue is harvested and immediately fixed in 10% buffered-formalin. At least 40 randomly chosen islets per mouse will be assessed from pancreas sections stained with H&E. Insulitis will be classified as: 0 (no insulitis); 1 (peri-insulitis—a weak peripheral inflammatory infiltrate that does not penetrate the islet tissue); 2 (moderate insulitis—an infiltrate <50% of the islet area); and 3 (strong insulitis—an infiltrate >50% of the islet area). Samples will be read blind. The submandibular glands, another site for the autoimmune process, will also be evaluated.

CD8⁺/TCR⁺ T cells as well as CD8⁺/TCR⁻ bone marrow cells have been referred to as FC (Kaufman, C. L., et al., Blood, 84:2436 (1994); Gandy, K. L., et al., Immunity, 11:579 (1999)). It is believed that CD8⁺/TCR⁻ FC are critical to engraftment in NOD mice. Removal of FC results in graft failure. CD8⁺/TCR⁺ T cells are supplemental in that they make space and promote engraftment.

Experiment 4: Do FC Promote Engraftment of HSC In NOD Mice

| | | | FC | | HSC | |
|---|---|---|---|---|---|---|
| | | FC | CELL | HSC | CELL | |
| GROUP | N | DONOR | DOSE | DONOR | DOSE | RECIPIENT |
| A | 6 | None | None | B10.BR | 10,000 | NOD |
| B | 6 | None | None | B10.BR | 20,000 | NOD |
| C | 6 | None | None | B10.BR | 30,000 | NOD |
| D | 6 | None | None | B10.BR | 40,000 | NOD |
| E | 6 | None | None | B10.BR | 50,000 | NOD |
| F | 6 | None | None | B10.BR | 10,000 | B10.BR |
| G | 6 | B10.BR | 10,000 | B10.BR | 10,000 | NOD |
| H | 6 | B10.BR | 20,000 | B10.BR | 10,000 | NOD |
| I | 6 | B10.BR | 30,000 | B10.BR | 10,000 | NOD |
| J | 6 | B10.BR | 40,000 | B10.BR | 10,000 | NOD |
| K | 6 | B10.BR | 50,000 | B10.BR | 10,000 | NOD |
| L | 6 | B10.BR | 10,000 | B10.BR | 20,000 | NOD |
| M | 6 | B10.BR | 20,000 | B10.BR | 20,000 | NOD |
| N | 6 | B10.BR | 30,000 | B10.BR | 20,000 | NOD |
| O | 6 | B10.BR | 40,000 | B10.BR | 20,000 | NOD |
| P | 6 | B10.BR | 50,000 | B10.BR | 20,000 | NOD |

Again, recipients will be conditioned with 1000 cGy TBI. Groups A-E will determine whether increasing numbers of HSC will engraft in allogeneic recipients without FC. Group F will control for the HSC sort itself: they will engraft in a syngeneic recipient. Groups G-K will establish the dose of FC required to enable engraftment of allogeneic HSC in NOD mice. In the initial experiments, two doses of HSC will be utilized in case more HSC are required for engraftment in NOD mouse recipients. Based on the fact that two to three times more bone marrow cells are required for engraftment in NOD mice, one would predict that two to three times more HSC will be required. If necessary, the HSC dose will be increased beyond 20,000 if engraftment is not detected at 20,000 HSC. These experiments will define the role of FC in establishing mixed chimerism in NOD mice.

As in previous experiments mice will be typed at 28 days and monthly thereafter. A WBC will be performed monthly. This requires only 20 microliters of blood and serves as an excellent indicator of engraftment. Animals will be monitored for glucosuria. At fixed time points (two months, four months, and six months), four animals per group will be euthanized and their pancreata examined for autoimmune status.

αβ-TCR⁺ and γβ-T cells have also been shown to “facilitate” engraftment. The next set of experiments will define the role of conventional T cells versus FC in engraftment in NOD recipients. When normal recipients are conditioned with 800 cGy of TBI, marrow depleted of T cells does not engraft unless γδ-T cells are added (Drobyski, W. R., et al., Blood, 89:1100 (1997)). This conditioning does not completely inactivate certain effector cells which mediate graft rejection (host-anti-donor alloreactivity) and that the αβ and/or γδ-T cells are required to neutralize those cells by providing space-making capacity. While αβ and γδ-T cells are not critical to HSC engraftment, they each contribute to the level of chimerism in a non-redundant fashion if FC are present (see preliminary results and progress report section.) Moreover, γδ-T cells contribute less significantly to GVHD compared with αβ-TCR⁺ T cells on a dose-dependent basis. One could look upon γδ-T cells as engraftment-enhancers in the presence of FC. Graft engineering to add a low level of γδ-T cells to FC plus HSC will allow engraftment at a reduced dose of conditioning. However, γδ-T cells will not function as FC in isolation with HSC in NOD recipients. The following experiments will answer that question and define the role of T cells in engraftment and chimerism in NOD mice. The final outcome in these experiments will dissect the contribution of CD8⁺/TCR⁻ FC from γβ-TCR⁺ T cells and αβ-TCR+/CD8⁺ T cells. An understanding of the mechanism of action and contribution of each cell type will allow a focused approach to establishing mixed chimerism in NOD mice with minimal recipient conditioning. CD8⁺/TCR⁻ FC provide an immunoregulatory trophic effect involving cell:cell contact to maintain the HSC in a self-renewing, primitive state while CD8⁺/TCR⁺ T cells and γβ-T cells provide space-making potential to alloreactive host cells. If this hypothesis holds true, approaches to target those alloreactive (host anti-donor) cells in the recipient microenvironment that are the targets of the donor veto cells and optimize FC dosing may be preferred over add-back of conventional donor-derived T cells, since they have the limitation that they produce GVHD. However, if T cells are critical to HSC survival, then they may be a necessary evil in BMT.

Experiment 5: Do Donor γδ-T Cells Provide Space-Making Capacity

| | | DONAR | γδ- | | | |
|---|---|---|---|---|---|---|
| | | (CD8⁺/ | T CELL | HSC | TBI | |
| GROUP | N | TCR⁻) | DOSE | DONOR | DOSE | RECIPIENT |
| A | 6 | B10.BR | — | B10.BR | 100 | NOD |
| B | 6 | B10.BR | 30,000 | B10.BR | 100 | NOD |
| C | 6 | — | 30,000 | B10.BR | 100 | NOD |
| D | 6 | B10.BR | — | B10.BR | 750 | NOD |
| E | 6 | B10.BR | 30,000 | B10.BR | 750 | NOD |
| F | 6 | — | 30,000 | B10.BR | 750 | NOD |
| G | 6 | B10.BR | — | B10.BR | 650 | NOD |
| H | 6 | B10.BR | 30,000 | B10.BR | 650 | NOD |
| I | 6 | — | 30,000 | B10.BR | 650 | NOD |
| J | 6 | B10.BR | — | B10.BR | 550 | NOD |
| K | 6 | B10.BR | 30,000 | B10.BR | 550 | NOD |
| L | 6 | — | 30,000 | B10.BR | 550 | NOD |

Groups A-C will define the role of CD8⁺/TCR⁻ FC and γδ-T-cells in HSC survival and self-renewal in fully ablated NOD mice. Groups A and B will probably exhibit durable engraftment while Group C will not. Survival in Group C will most likely be prolonged due to the function of committed progenitors but that HSC will not remain self-renewing beyond approximately six months. Groups D-L will define the role of HSC plus FC alone (Groups D, G, and J) versus HSC plus γδ-T cells plus FC (Groups E, H, K) versus HSC plus γδ-T cells alone Groups F, I, and L) in the context of reduced conditioning. γδ-T cells will probably not sustain HSC engraftment in NOD recipients but that they will enhance chimerism at a lower TBI dose if FC are present. Recipients will be monitored for GVHD clinically. Daily weights will be recorded and animals with diarrhea or skin changes will undergo histologic evaluation.

If αβ-TCR⁺ cells are depleted from the B10.BR donor marrow, NOD recipients conditioned with 950 cGy TBI do not engraft. Whether this is purely due to graft rejection or rather that αβ-TCR⁺ T cells also function as FC in NOD recipients has not been resolved. In normal recipients, Gandy et al. demonstrated that CD8^{total} cells resulted in higher levels of chimerism compared with CD8⁺/TCR⁻ cells alone. However, they did not show that CD8⁺/TCR⁺ cells alone could promote long-term survival of HSC in allogeneic recipients. They only demonstrated that CD8^{total} FC (αβ-TCR⁺ plus αβ-TCR⁻) resulted in higher levels of donor chimerism at 30 days compared with CD8⁺/TCR⁻ FC.

Experiment 6: Determine Whether αβ-T Cells Can Function as FC

| | | | αβ-T | | |
|---|---|---|---|---|---|
| | | FC DONOR | CELL | TBI | |
| GROUP | N | (CD8⁺/TCR⁻) | DOSE | DOSE | RECIPIENT |
| A | 6 | 30,000 | — | 1000 | NOD |
| B | 6 | 30,000 | 30,000 | 1000 | NOD |
| C | 6 | — | 30,000 | 1000 | NOD |
| D | 6 | 30,000 | — | 950 | NOD |
| E | 6 | 30,000 | 30,000 | 950 | NOD |
| F | 6 | — | 30,000 | 950 | NOD |
| G | 6 | 30,000 | — | 850 | NOD |
| H | 6 | 30,000 | 30,000 | 850 | NOD |
| I | 6 | — | 30,000 | 850 | NOD |
| J | 6 | 30,000 | — | 750 | NOD |
| K | 6 | 30,000 | 30,000 | 750 | NOD |
| L | 6 | — | 30,000 | 750 | NOD |

Groups A-C are fully ablated. The majority of residual alloreactive host anti-donor “radioresistance” cells will therefore be minimized. With decreasing doses of TBI, we suspect that T cells will play an increasingly more important role in engraftment in NOD recipients if they function by a veto mechanism to neutralize residual host cells for engraftment. If they function as FC, then even at 1000 cGy TBI they will be required.

The factors that influence engraftment are multifactorial. Both host and donor elements can be manipulated to influence outcomes. By tipping the balance in favor of the donor marrow composition and cell dose, one can achieve engraftment with reduced conditioning. For example, megadose stem cell therapy allows engraftment in partially conditioned recipients at a significantly reduced rate of TBI (Aversa, F., et al., Blood, 84:3948 (1994); Sykes, M., et al., Nature Medicine, 3:783 (1997); Fuchimoto, Y., et al., J. Clin. Invest., 105:1779 (2000); Colson, Y. L., et al., J. Immunology, 155:4179 (1995)). In our own model for partial conditioning, when the allogeneic bone marrow cell dose was doubled, the TBI dose could be reduced by 50%. When FC are reduced in number, more TBI is required for engraftment to be achieved.

The FC and HSC that are mobilized into the peripheral circulation are functional to radioprotect from irradiation-induced aplasia. Interestingly, the HSC that remain behind in the marrow compartment are expanded in number but are functionally impaired, even for syngeneic engraftment. The mechanism for this observation is outside the scope of this application but it highlights the importance of a functional (in vivo) evaluation for FC and HC function.

Bone marrow donors will receive 10 μg of FL plus 7.5 μg of G-CSF per day by subcutaneous injection for 10 days. At day 11 the animals will be exsanguinated and HSC and FC from the peripheral blood will be sorted separately in the standard fashion. The optimum number of HSC determined in experiment 7 will be administered per recipient. FC and HSC will be mixed in varying ratios as indicated in Experiment 8 prior to infusion. It is believed that by increasing the FC dose, the minimum TBI dose required for engraftment will be reduced significantly.

Experiment 7: Can Conventional CD8⁺/TCR⁺ T Cells Also Function as FC

| | | | CD8⁺/TCR⁺ | |
|---|---|---|---|---|
| GROUP | N | FC | T-Cells | HSC |
| A | 6 | — | 10,000 | + |
| B | 6 | — | 20,000 | + |
| C | 6 | 30,000 | + | |
| D | 6 | 10,000 | 10,000 | + |
| E | 6 | 20,000 | 20,000 | + |
| F | 6 | 30,000 | 30,000 | + |
| G | 6 | 10,000 | — | + |
| H | 6 | 20,000 | — | + |
| I | 6 | 30,000 | — | + |

Recipients will be conditioned with 1,000 cGy TBI. Groups A-C will answer whether CD8⁺/TCR⁺ T cells can facilitate HSC engraftment on their own. Groups D-F will evaluate the role of T cells on level of chimerism when FC are present, compared to FC alone (Groups G-I). As the role for FC versus conventional T cells is defined, strategies to optimize engraftment yet minimize morbidity will emerge. FC are critical for HSC engraftment in NOD recipients and that T cells are only supplemental.

Experiment 8: Do Cytokine-Potentiated FC Allow Engraftment With Reduced Conditioning

| | | | FC | | HSC | |
|---|---|---|---|---|---|---|
| GROUP | N | TBI | DONOR | FC DOSE | DONOR | RECIPIENT |
| A | 6 | 1000 | B10.BR | 30,000 | B10.BR | NOD |
| B | 6 | 1000 | B10.BR | 45,000 | B10.BR | NOD |
| C | 6 | 1000 | B10.BR | 60,000 | B10.BR | NOD |
| D | 6 | 750 | B10.BR | 30,000 | B10.BR | NOD |
| E | 6 | 750 | B10.BR | 30,000 | B10.BR | NOD |
| F | 6 | 750 | B10.BR | 60,000 | B10.BR | NOD |
| G | 6 | 650 | B10.BR | 30,000 | B10.BR | NOD |
| H | 6 | 650 | B10.BR | 45,000 | B10.BR | NOD |
| I | 6 | 650 | B10.BR | 60,000 | B10.BR | NOD |
| J | 6 | 550 | B10.BR | 30,000 | B10.BR | NOD |
| K | 6 | 550 | B10.BR | 45,000 | B10.BR | NOD |
| L | 6 | 550 | B10.BR | 60,000 | B10.BR | NOD |
| M | 6 | 750 | B10.BR | 30,000 | B10.BR | NOD |

Titrating doses of irradiation (Groups A-C; D-F, G-I, and J-L) will be administered. The optimal HSC dose established in experiment 7 will be utilized. FC dose will be titrated in that context from 30,000 (Groups A, D, G and J) to 45,000 (Groups B, E, H and K) to 60,000 (Groups C, F, I and L). The donor for Group J will not be cytokine treated. It is hypothesized that by increasing the FC dose mixed chimerism will be established with a lower dose of TBI. Methods to expand FC and optimize biologic activity are part of a nonoverlapping proposal. However any improvements in the FC expansion will be tested within this model in the NOD mouse.

Experiment 9: Are NOD Mixed Chimeras Tolerant to Islet Allografts.

NOD mice reject islet allografts from B10.BR donor mice (median survival time 17 days). After we have defined the optimal composition of the donor marrow inoculum is define, an evaluation of whether NOD chimeras will accept simultaneous and/or sequential islet allografts yet remain competent to reject MHC-disparate donor grafts will be performed. Obviously, the simultaneous islet transplant most closely mimics the clinical setting for cadaveric organ transplantation, while the sequential approach would be clinically viable only with live organ donation (i.e. for kidney grafts). On day +1 following BMT (simultaneous) or day 29 following BMT (sequential) an islet transplant from one of two sources will be performed: donor-specific (B10.BR, H2K^{k}) or MHC-disparate third party (B10, H2 Kb). Grafts will be placed under the left renal capsule. NOD mice will not be allowed to become diabetic, nor will diabetes be induced in order to avoid the expense of monitoring and housing of the animals. Grafts will be removed by transplant nephrectomy at 1 month, 4 months, 8 months, and 12 months post islet transplant (n=4/group) and evaluated histologically for evidence of 1) rejection; 2) insulin production; 3) recurrent insulitis. All grafts will be read blind. The native pancreata and salivary glands will also be harvested from the chimeras. Age-matched controls that are conditioned but do not receive a BMT will be evaluated for comparison (4 per time point) to evaluate the influence of the conditioning alone on induction of tolerance. Evidence for persistent systemic autoimmunity will likely appear. Chimerism will induce donor-specific tolerance to islet allografts as well as re-introduce tolerance to self-autoantigens by reversing the autoimmune process.

Host factors play a major role in regulating engraftment. Until recently, it was believed that complete recipient ablation was a requisite to durable engraftment of allogeneic HSC. The 10% mortality associated with full ablation has prevented the application of HSC chimerism to induce tolerance and reverse the autoimmune process in type I diabetes. A number of approaches have been developed to establish chimerism through partial conditioning. By combining cytoreduction with immunosuppression to inactivate residual host-anti-donor reactive cells, engraftment can be achieved with minimal manipulation of the recipient.

In normal mice, conditioning of the recipient with post-transplant cyclophosphamide (day +2) plus peri-transplant ALG allows engraftment to be achieved with only 200 cGy TBI. It was believed that the cyclophosphamide was eliminating alloreactive host cells in the host. If cyclophosphamide is given on day −2 or −1, engraftment does not result. A fascinating observation emerged when a partial conditioning approach was applied to NOD recipients: more conditioning is required for engraftment in NOD mice and a relative alloresistance to engraftment is present. Moreover, the NOD mouse as a recipient is relatively radioresistant, an observation that supports a role for defective apoptosis. A similar alloresistance to engraftment has been recognized in other animal models for autoimmunity and in humans who undergo a BMT for autoimmune aplastic anemia.

### Example 3

### Characterize Which Cells in the Nod Microenvironment Influence Engraftment

A technique to evaluate the effect of partial conditioning on various cellular in the host microenvironment using flow cytometry has been developed. This approach is very informative in normal mouse recipients to identify which cells in the recipient contribute to alloresistance to engraftment. Conditioning of normal recipients with 600 cGy TBI allows reliable engraftment of allogeneic BMC (Colson, Y. L., et al., J, 157:2820 (1996)). Cells present at 500cGy TBI and removed by 100 cGy higher were evaluated. These would be the putative cell types that must be targeted for engraftment to result. Normal B10.BR or B10 mice were conditioned and the marrow harvested at least 6 hours after treatment to most closely resemble the mouse model for BMT. In normal recipients, conventional T cells as well as T/NK cells must be eliminated for engraftment to occur (manuscript in preparation). B cells (B220⁺) cells also disappear even after a very low dose of irradiation.

Experiment 1: Determine Which Host Marrow Cell Types are Removed by Conditioning.

When NOD recipients are conditioned with 750 cGy TBI, they engraft with allogeneic bone marrow (Li, H., et al., J. Immunology, 156:380 (1996); Seung, E., et al., Blood, 95:2175 (2000)). At doses ≦650, engraftment does not occur without additional recipient pretreatment (i.e. anti-CD8 mAb). Mice will be conditioned with 750 or 650 cGy TBI. Marrow, thymocytes, and splenocytes will be harvested on day +1 relative to the conditioning. The critical cellular subsets for engraftment will be enumerated and compared. Theoretically, those cell populations present at 650 but absent at 750 are the most important effectors for alloresistance.

Experiment 2: Which Host Marrow Cell Types Are Removed by Conditioning

| CELL TYPE | STAIN 1-FITC | STAIN 2-PE | STAIN 3-APC |
|---|---|---|---|
| FC | Aβ-TCR⁻ plus γδ TCR⁻ | CD8⁺ | CD3∈⁺ versus |
| HSC | c-kit⁺ | Sca-1⁺ | Lin⁻ |
| T Cell | αβ-TCR⁺ | CD8⁺ | — |
| (cytotoxic) | | | |
| T Cell (helper) | αβ-TCR⁺ | CD4⁺ | — |
| T Cell | Γδ | — | — |
| NK Cell | ASGM-1⁺ | DX5⁺ | — |
| T/NK Cell | DX5⁺ | αβ-TCR⁺ | — |
| B Cell | B220⁺ | — | — |

This approach will serve as a useful guide to focus on certain putative effector cells to target in vivo with antibody preconditioning.

In normal mice, αβ and γδ host T cells contribute in a non-redundant fashion to alloresistance to engraftment. Mice deficient in production of αβ and γδ-T cells (βδ KO) engraft without TBI or cyclophosphamide at all. It is believed that conventional T cells in NOD recipients play a major role in resistance to allogeneic engraftment and that by targeting these cells the minimum TBI dose for engraftment will be able to be substantially reduced.

In Experiment 3, below, it is determined whether an increased dose of FC will allow engraftment with a decreased TBI dose. The cell dosing utilized in these experiments will be determined by the prior experiments (1 and 7). The conditioning will be titrated down from the established partial conditioning model (750 cGy TBI).

Experiment 3: Will Augmenting FC Dose Allow Reduction of TBI

| GROUP | N | TBI | FC | HSC |
|---|---|---|---|---|
| A | 6 | 750 | 30,000 | 10,000 |
| B | 6 | 650 | 30,000 | 10,000 |
| C | 6 | 650 | 40,000 | 10,000 |
| D | 6 | 650 | 50,000 | 10,000 |
| E | 6 | 550 | 30,000 | 10,000 |
| F | 6 | 550 | 40,000 | 10,000 |
| G | 6 | 550 | 50,000 | 10,000 |

It is believed that as the TBI dose is decreased from 750 (control; Group A) to 650 (Groups B-D) and 550 (Groups E-G), engraftment will occur with augmentation of the FC dose. As in previous experiments, recipients will be followed for chimerism and development of diabetes. Controls will be conditioned but not transplanted.

In continuing experiments, strategies to potentiate the biologic effect of FC will be pursued. Hematopoietic growth factors play a major role in HSC survival, self-renewal, and function. Data strongly suggests that FC are also influenced by hematopoietic growth factors. Treatment of normal mice with FLT-3 Ligand (FL) in vivo results in a significant increase in biologically active FC (Lampeter, E. F., et al., Lancet, 341:1243 (1993)). The gene-chip expertise will allow a determination of which additional cytokine and growth factor receptors are present on FC. For this purpose, the Affymetrix oligonucleotide microarray technology was selected because its use of multiple probes ensures greater signal-to-noise ratios and fewer ‘false-positives’ from cross-hybridization than other microarray methods. Each gene tested for expression level is analyzed using 20 pairs of 25-mer oligonucleotides. Under the stringent hybridization conditions used, mRNAs with sequences that are matched to those of the synthetic oligonucleotides on the chips produce hybridization signals that are proportional to the steady state level of transcript present in the sample. Because the probes are multiply redundant for each gene, outlier rejection and averaging of signal intensities can be used to increase the accuracy of RNA quantitation. Affymetrix reports that their technology allows for an absolute quantitative accuracy of + or −2-fold changes in transcript levels, with a dynamic range of up to four orders of magnitude (Lipshutz, R. J., et al., Nat. Genet., 21:20). The Affymetrix mu 11K gene-chip reports expression levels of 11,000 mouse genes, including at least 248 that correspond to growth factors or their receptors. The mu 11K gene-chip will be used for triplicate measurements of steady-state mRNA levels in sorted populations of FC to determine which receptors may be expressed. At least 5×10⁶ cells will be needed to produce the 5 μg of total RNA used in each hybridization analysis, for a total of 15×10⁶ cells (or 15 μg total RNA) needed for three replicate experiments.

One potential pitfall to using gene chip arrays is that one can be distracted by the volume of data obtained. A second potential pitfall is reproducibility. Conditions must be carefully controlled and experiments repeated at least four times to confirm reproducibility of the findings. The gene chip technology can be a powerful research tool if used in a focused, hypothesis-driven approach. In this case which cytokine receptors are present on FC will be defined with the hypothesis that those growth factors identified could provide a method for expansion and potentiate activity.

FC will be cultured in a mixture of cytokines whose receptors are expressed on FC for 72 hours prior to administration. FC will then be transplanted with 10,000 purified HSC into ablated NOD recipients. FL results in a significant expansion of FC in vivo (Lampeter, E. F., et al., Lancet, 341:1243 (1993)). Moreover, FL-expanded FC exhibit enhanced biologic activity in vivo. By combining FL with other cytokines for which receptors are identified on FC, strategies to decrease the conditioning required for engraftment in NOD recipients will be developed. As in previous experiments cytokine-expanded FC will be transplanted with genetically matched HSC and the minimum TBI dose for engraftment identified.

APC from the NOD mouse and from humans with type I diabetes possess a number of well-described defects in development, Class II expression, and function (Naji, A., et al., Annals of Surgery, 194:328 (1981); Theofilopoulos, A. N., et al., J. Experimental Medicine, 162:1 (1985); Serreze, D. V., et al., Diabetes, 37:252 (1988); Ikehara, S., et al., Proceed. Natl. Acad. Sci. USA, 82:2483 (1985)) (Marmont, A. M., et al., Forum, 5:24 (1995)) which could be responsible for impaired clonal deletion of potentially autoreactive T cells. The FC could induce tolerance in one of two ways. It could itself be a tolerance-inducing cell that migrates to the thymus and/or periphery to mediate deletion of potentially autoreactive T cells. Conversely, it could promote survival of HSC, which in turn produce progeny to restore normal clonal deletion.

Experiment 4 will determine whether FC are found in the thymus. Donors congenic for all histocompatibility antigens but disparate for the CD45 isoforms Ly5.1 and Ly5.2 will be utilized. This is not an antigenic disparity, nor is it a histocompatibility disparity. Monoclonal antibodies exist against each isoform. Briefly, NOD recipients will be irradiated with the optimal TBI dose determined in AIM I and transplanted with purified FC and HSC in one of 4 combinations.

Experiment 4: DO Conventional Host T Cells Mediate Alloresistance

| GROUP | N | mAb 1 | mAb 2 | mAb 3 | cGy |
|---|---|---|---|---|---|
| A | 6 | — | — | — | 750 |
| B | 6 | Anti-αβ TCR | — | — | 650 |
| C | 6 | Anti-αβ TCR | — | — | 550 |
| D | 6 | — | anti-γδ TCR | — | 650 |
| E | 6 | — | anti-γδ TCR | — | 550 |
| F | 6 | Anti-αβ TCR | anti-γδ TCR | — | 650 |
| G | 6 | Anti-αβ TCR | anti-γδ TCR | — | 550 |

Mice will be conditioned with varying doses of irradiation and transplanted with the composition of marrow identified in Example 1 to optimize engraftment in the absence of the host anti donor reactivity (full ablation). Animals will be typed monthly for multilineage chimerism. If 100% of animals engraft at the lowest level of TBI listed, titration will continue to decrease until a point when engraftment does not occur is identified. The optimal approach will be with combined anti-αβ TCR plus anti-γδ TCR mAb preconditioning.

### Experiment 5: Depletion Kinetics of Monoclonal Antibodies in Vivo to Determine Optimal Dose and Time of Administration

For each of the antibodies to be tested, dose-titrations of purified antibody will be performed, starting with the dose that has been established to be effective in normal mice, to determine how much antibody is required for in vivo depletion of that target population in the NOD mouse. Injection of 1001g of a depleting anti-CD8 mAb on day −3 completely depletes CD8⁺ cells from the circulation of normal B10 or B10.BR mice. Confirmation that the cells were depleted and not just coated with antibody occurred by staining with a goat-anti-isotype-species of origin for mAb sandwich antibody. In striking contrast, a total of four times more of the same anti-CD8 mAb (400 μg) is required to completely remove CD8 T cells from the peripheral blood in NOD mice. For each antibody to be tested, animals will be injected with 100 μg of monoclonal antibody on day −3, then peripheral blood lymphocytes (PBL) will be collected from the recipients and evaluated for adequacy of depletion as well as cotating on day −3, −2, 1, and 0 with respect to the timing for irradiation and BMT. For each mAb, two NOD mice will be conditioned with antibody, monitored daily by PBL, then euthanized at day 0. B10.BR mice will be treated in a similar fashion as controls. If complete depletion is not noted, a higher dose of antibody on day −3 will be administered to the next 2 animals and monitor as before. On day 0 we will euthanize the mAb-conditioned recipients. Bone marrow, splenocytes, and lymph node cells will be analyzed for coating or adequacy of cellular depletion using: 1) goat-anti-species of origin-Immunoglobulin-FITC (to detect coating of residual cells that were not eliminated); and 2) saturating levels of the mAb utilized for depletion-FITC. With this approach the dosing and timing for in vivo depletion with each individual antibody will be optimized.

One potential pitfall to these studies is that some mAb are nondepleting but still exert an influence on conditioning (Saitovitch, D., et al., Transplantation, 61:1642 (1996)). In that case, increasing the dose of antibody will not result in more efficient depletion of the target population but we will reach saturation kinetics with respect to staining profile for antibody coating. Therefore, while it will be important to know whether an mAb is depleting versus non-depleting in vivo, an antibody will not be excluded because it is not depleting until tested in vivo.

It was decided to increase the antibody dose rather than give antibody on different days because it was determined by ELISA that mAb injected in mice in vivo remains in the circulation for at least 3 weeks. Therefore, it is the total dose of antibody that is important rather than repeated injections temporally spaced closely together. The optimum dose of purified mAb will be injected intraperitoneally in vivo with timing for each antibody established based upon where the maximum effect for in vivo depletion or coating of target cells was achieved at this time point.

Experiment 6: Are Partially Conditioned NOD Chimeras Functionally Tolerant to Donor Alloantigens

If tolerance can be established in NOD mice using partial conditioning, it will be important in two respects clinically: 1) to induce tolerance for islet allografts after diabetes has developed; and 2) to establish chimerism prior to the development of insulin-dependence, to re-introduce self-tolerance. It is important to test chimeric recipients for evidence of functional tolerance in vivo and in vitro.

Functional tolerance in vitro using MLR and CML assays is evaluated. The MLR tests for CD4⁺ T-cell tolerance (proliferation results in thymidine uptake) and the CML tests for CD8⁺ (cytotoxic) tolerance to donor alloantigens (lysis of ⁵¹ CR labeled targets results in release of ⁵¹CR into the supernatant). Splenic lymphocytes from 1) NOD chimeras; 2) conditioned yet untransplanted NOD controls; and 3) unmodified age-matched NOD mice will be co-cultured with irradiated (2500 cGy) syngeneic (to control for cell dose), donor (B10.BR), or MHC-disparate third party alloantigen in one-way CML and MLR assays. Chimeras will be tested at 2 months and at 6 months post transplantation to confirm the presence and durability of functional tolerance in vitro.

It has been previously demonstrated that targeting of recipient CD8⁺ cells allows the TBI dose to be reduced to 700 cGy in NOD recipients. This CD8⁺ cell population is comprised of conventional T cells plus a TBI but not cyclophosphamide-sensitive cellular subset that is CD8⁺/CD3ε⁺/NK1.1⁺ in normal mouse recipients. By combining anti-CD8 treatment with removal of host αβ plus γδ T cells the TBI dose will be further reduced by combining agents that are graft-promoting that target different cell populations.

Experiment 7: Does Targeting of CD8⁺ Plus T Cells Reduce the TBI Dose

| GROUP | N | MAB 1 | MAB 2 | MAB 3 | TBI |
|---|---|---|---|---|---|
| A | 6 | anti-CD8 | αβ TCR | — | 700 |
| B | 6 | anti-CD8 | — | γδ TCR | 700 |
| C | 6 | anti-CD8 | αβ TCR | γδ TCR | 600 |
| D | 6 | anti-CD8 | αβ TCR | γδ TCR | 500 |
| E | 6 | CD8 | — | — | 700 |

Group E is the control for the established model. In Groups A-D the minimum TBI dose required for engraftment of allogeneic marrow will be identified. Recipients will be typed for chimerism at 28 days and monthly thereafter. As in previous experiments, once the minimum TBI dose required is established recipients will be conditioned but not transplant them to confirm that the approach is not ablative to a point of aplasia. Recipients will be tested for donor-specific tolerance in vitro by MLR and CML assays and in vivo by islet transplantation.

The fundamental underlying hypothesis for this aim is that there are specific cells in the recipient with specific radiation-sensitivity that must be disabled for engraftment of allogeneic marrow to occur. In light of the fact that a low dose of TBI (˜250 cGy) is sufficient for syngeneic marrow to engraft and higher levels are required for allogeneic marrow to engraft, it is hypothesized that there are two requirements for engraftment to occur in allogeneic recipients: (1) vacant niches, and (2) control of the alloreactive host cells. It is also believed that the alloreactive component is more dominant than the niche component, especially in NOD mice. Because splenocytes from NOD mice are in a chronically more activated state it is likely that the cytokine storm that accompanies in autoimmune state supports alloreactivity as well.

Activation of T-lymphocytes requires co-stimulatory molecules. The CD40-CD154 (CD40-CD40 Ligand) and B7-CD28 T cell co-stimulatory pathways play a critical role in T cell activation via APC interaction (Rossini, A. A., et al., Physiol Rev., 79:99 (1999); Thomson A. W., et al., Immunol. Today, 20:27 (1999); Larsen C. P., et al., Curr. Opin. Immunol., 9:641 (1997)). It is hypothesized that in vivo blockade of these molecules will allow engraftment at a reduced TBI dose by preventing effector function of the host versus donor response. Administration of anti-CD40L mAb to NOD recipients allowed engraftment of allogeneic marrow with as low as 600 cGy of TBI in 100% of recipients. Similarly, pre-treatment of NOD recipients with anti-CTLA-4 mAb also decreased the minimum levels of TBI for engraftment. In the next set of experiments in vivo targeting of T cells is combined with co-stimulatory blockade. The rationale is that the co-stimulatory blockade will disable residual T cells not effectively removed by the in vivo mAb pre-treatment. The anti-TCR mAb and anti-CD8 mAb will be administered at the time point prior to BMT and TBI that provides most efficient removal of the target cell. For anti-CD8 mAb we know that the mAb has its greatest effect when administered 3 days prior to BMT. The anti-CD 154 mAb will be administered on day 0 in the morning 6 hours prior to bone marrow infusion in the afternoon. Twelve-week old NOD mice with active autoimmunity will be conditioned with varying doses of irradiation after pre-treatment with anti-CD8 plus anti-CD154 (Groups A-C); anti-CD154 plus anti-αβ TCR (Groups D-F); anti-CD154, CD8 and αβ-TCR (Groups G-I).

Experiment 8: Does Co-Stimulatory Blockade in Combination With Targeting host CD8⁺ and T Cells Further Reduce TBI

| GROUP | N | MAB | MAB | MAB | TBI |
|---|---|---|---|---|---|
| A | 6 | anti-CD154 | anti-CD8 | — | 500 |
| B | 6 | anti-CD154 | anti-CD8 | — | 400 |
| C | 6 | anti-CD154 | anti-CD8 | — | 300 |
| D | 6 | anti-CD154 | — | anti-αβ | 500 |
| E | 6 | anti-CD154 | — | anti-αβ | 400 |
| F | 6 | anti-CD154 | — | anti-αβ | 300 |
| G | 6 | anti-CD154 | anti-CD8 | anti-αβ | 500 |
| H | 6 | anti-CD154 | anti-CD8 | anti-αβ | 400 |
| I | 6 | anti-CD154 | anti-CD8 | anti-αβ | 300 |
| J | 6 | anti-CD154 | | | |

As in previous experiments, recipients will be typed monthly for chimerism. Animals will be monitored for development of diabetes. At 1, 2, 4, 8 and 12 months four recipients per conditioning group in those where engraftment occurred in 100% of recipients will be euthanized and the tissue examined for GVHD (tongue, gut) and the status of autoimmunity (pancreata, submandibular glands). Finally, splenocytes will be harvested from these animals and tested in vitro in MLR and CML assays. Through these studies the effect of chimerism is evaluated on autoimmunity and alloreactivity.

Mice genetically modified to resist diabetes, insulitis, or both, have provided valuable information on linkage to disease resistance and the genetics of diabetes. The mechanism by which mixed chimerism reverses insulitis and diabetogenesis in NOD mice is not understood. The mechanism of reversal by comparing chimeric NOD mice to those that have been rendered diabetes-resistant via congenic breeding to disease-resistant strains of mice such as C57BL/6 and C57BL/10 is evaluated. Wicker and others have defined several genetic loci that, when bred from C57BL to NOD mice, reduce the incidence of diabetes from 80-90% to 3-7% in female mice (Wicker, L. S., et al., Annu. Rev. Immunol., 13:179-200:179 (1995)). The resistance loci fall into two categories: (1) genes that reduce both diabetes onset as well as insulitis, and (2) genes that reduce the onset of diabetes without reducing insulitis. Loci that reduce both insulitis and diabetes include insulin-dependent disease locus 1 (1) and the linked resistance alleles 3, 10, 17, 18. The 1 allele of NOD mice expresses MHC H-2^{g7} and is well known as being required for disease onset in experimental mice. This requirement occurs presumably because MHC II I-A^{g7} permits presentation of β cell antigens to T cells with sufficient affinity to allow antigen-dependent cytolysis of islet cells. The linked loci 3, 10, 17, 18 are most protective when expressed in combination and are largely uncharacterized, although 3 has tentatively been identified in NOD mice as an allele of the IL-2 gene (Podolin, P. L., et al., Cytokine, 12:477 (2000)). Hence, genes that are protective because they reduce both insulitis and diabetes may function by limiting the early steps of diabetogenesis such as cellular recruitment to pancreatic islets and proliferation in response to antigen.

The second class of genes that confer resistance to diabetes appear to function by limiting the intermediate steps of diabetogenesis, such as destruction of pancreatic islets after cellular infiltration has occurred. The best characterized locus in this category, 9, is a genetic interval that includes molecular variants of CD30, TNF receptor II, and CD137 (Lyons, P. A., et al., Immunity, 13:107 (2000)). Notably, NOD mice with the 9 locus of C57BL/10 mice (NOD.B10 9) have fully infiltrating insulitis, but do not progress to diabetes. NOD.B10 9 mice have infiltrating lymphocytes that express CD30 and secrete IL-4, while NOD mice have infiltrating lymphocytes that secrete islet destructive cytokines such as TNFα and IFNγ (Lyons, P. A., et al., Immunity, 13:107 (2000)). Hence, 9 may be protective because it promotes T cell differentiation to a T_{H}2 or other regulatory phenotype from the T_{H} 1 expression profile of inflammatory cytokines. These analyses of genetic resistance loci and their resulting phenotypes provides an important description of how a step-wise progression to diabetes might occur. Through these studies it will be determined whether the hematopoietic abnormalities in NOD mice are linked to the insulitis and diabetes loci or are independent from them. A better understanding of the mechanism by which chimerism reverses the autoimmune process will allow more focused approaches to optimize the outcome and reverse the disease process.

Mouse Strains Congenic for Diabetes Resistance Genes: Influence of Genetic Linkage on Insulitis and Diabetes

| | | | | TENDENCY |
|---|---|---|---|---|
| | | | | TO |
| MOUSE | | | RADIORESI | TCD GRAFT |
| STRAIN | INSULITIS | DIABETES | STANCE | FAILURE |
| NOD | +++ | +++ | +++ | +++ |
| NOD.B10 1 | −−− | −−− | ? | ? |
| NOD.B10 3, | −−− | −−− | ? | ? |
| NOD.B10 9 | +++ | −−− | ? | ? |

NOD mice exhibit a number of hematopoietic abnormalities. Whether these defects are linked to the loci that influences diabetes versus insulitis versus disease-resistance has not been examined. Because diabetes-prone NOD mice show a paucity of FC cells, we will first examine whether each of these strains have FC in their marrow (EXPERIMENT 9). Marrow will be harvested from each strain (n=4 per group) at twelve weeks of age and analyzed for FC, HSC, and conventional T cells. B10.BR mice will be prepared as controls.

Experiment 9: Do NOD Congenic Mice Have FC

| MOUSE STRAINS | CELLS TO BE ENUMERATED |
|---|---|
| TO BE TESTED | IN EACH STRAIN |
| NOD | FC (CD8^{+/}TCR⁻/CD3∈⁺) |
| NOD.B10 (3, 10, 17, 18) | HSC |
| NOD.B10 9 | CD8⁺/TCR⁺ |
| B10.BR | CD4⁺/TCR⁺ |

In EXPERIMENT 10, the radioresistance of each congenic NOD strain will be evaluated. It is possible that radioresistance will be linked only to the full diabetic phenotype (NOD), or that it may be linked to insulitis alone (9) or disease resistance (3, 10, 17, 18) genes. Animals will be conditioned and not given marrow (Groups A, B, E, F, G, K, L, and M) or transplanted with 15×10⁶ allogeneic bone marrow cells. The group that is conditioned alone will define the radiation sensitivity of each congeneic strain. The transplanted group will receive a cell dose that allows reliable engraftment in normal but not NOD 1 mice. If engraftment occurs at this cell dose at any TBI dose, it will indicate a lesser resistance to engraftment. The TBI dose at which engraftment occurs will also be informative.

Experiment 10: Determine Genetic Linkage of Relative Radioresistance.

| GROUP | N | RECIPIENT | DONOR | TBI DOSE |
|---|---|---|---|---|
| A | 4 | NOD | — | 850 |
| B | 4 | NOD | — | 750 |
| C | 4 | NOD | B10.BR | 750 |
| D | 4 | NOD | B10.BR | 650 |
| E | 4 | NOD.B10 9 | — | 850 |
| F | 4 | NOD.B10 9 | — | 750 |
| G | 4 | NOD.B10 9 | — | 650 |
| H | 4 | NOD.B10 9 | B10.BR | 750 |
| I | 4 | NOD.B10 9 | B10.BR | 650 |
| J | 4 | NOD.B10 9 | B10.BR | 550 |
| K | 4 | NOD.B10 (3, 10, 17, 18) | — | 850 |
| L | 4 | NOD.B10 (3, 10, 17, 18) | — | 750 |
| M | 4 | NOD.B10 (3, 10, 17, 18) | — | 650 |
| N | 4 | NOD.B10 (3, 10, 17, 18) | B10.BR | 750 |
| O | 4 | NOD.B10 (3, 10, 17, 18) | B10.BR | 650 |
| P | 4 | NOD.B10 (3, 10, 17, 18) | B10.BR | 550 |

As in previous experiments, mice will be typed at 28 days and monthly thereafter and followed for development of diabetes.

In EXPERIMENT 11, the sensitivity of each mouse strain TCD graft failure is evaluated. It is believed that an impaired potential for engraftment will segregate with one or more of the genetic loci. B10.BR mice will be utilized as donors. Recipients will be conditioned with 1000 cGy TBI. The marrow will be depleted of T cells using RAMB.

Experiment 11: What Is the Genetic Linkage of Tendency to TCD Graft Failure

| GROUP | N | RECIPIENT | DONOR | CELL DOSE |
|---|---|---|---|---|
| A | 4 | NOD | B10.BR | 15 × 10⁶ TCD |
| B | 4 | NOD | B10.BR | 30 × 10⁶ TCD |
| C | 4 | NOD | B10.BR | 45 × 10⁶ TCD |
| D | 4 | NOD | B10.BR | 45 × 10⁶ |
| | | | | untreated |
| E | 4 | NOD.B10 Idd 9 | B10.BR | 15 × 10⁶ TCD |
| F | 4 | NOD.B10 Idd 9 | B10.BR | 30 × 10⁶ TCD |
| G | 4 | NOD.B10 Idd 9 | B10.BR | 45 × 10⁶ TCD |
| H | 4 | NOD.B10 Idd 9 | B10.BR | 45 × 10⁶ |
| | | | | untreated |
| I | 4 | NOD.B10 Idd (3, 10, 17, 18) | B10.BR | 15 × 10⁶ TCD |
| J | 4 | NOD.B10 Idd (3, 10, 17, 18) | B10.BR | 30 × 10⁶ TCD |
| K | 4 | NOD.B10 Idd (3, 10, 17, 18) | B10.BR | 45 × 10⁶ TCD |
| L | 4 | NOD.B10 Idd (3, 10, 17, 18) | B10.BR | 45 × 10⁶ |
| | | | | untreated |

Groups A-C are controls. They will exhibit failure of engraftment. Group D will engraft. Animals will be typed for chimerism at 28 days and monthly thereafter. They will also be monitored for diabetes. At selected time points (1 month, 2 months, 4 months, 6 months, 12 months) animals will be euthanized and the pancreata and salivary glands harvested and evaluated for insulitis and autoimmunity.

After the genetic linkage for the sensitivity is established for TCD graft failure and the relative radioresistance to conditioning, mixed chimeras will be prepared using each of the 3 strains as a recipient. Animals will be monitored as in previous experiments and the percentage donor versus host chimerism will be followed. The tendency of NOD to convert from mixed chimeras to be nearly 100% donor may be influenced by whether that strain is capable of producing functional endogenous FC, and that those strains that have FC will remain stable mixed chimeras.

Through these experiments it will be determined whether the disease-susceptibility characteristics are linked to the alloresistance phenotype observed in NOD mice and better understand the stepwise progression to disease.

### Example 4

### Define the Mechanism by Which Mixed Chimerism Restores Self-Tolerance in NOD Mice

NOD mice exhibit a relative radioresistance. Most radioresistance is due to thwarting of checkpoints in the regulation of the cell cycle following radiation damage. Irradiation damages the DNA in a cell. In normal circumstances, the cell stops to check on the status of the DNA and decides to stop and repair the damage or destroy the cell. This is regulated by genes in the Bcl family. Resistance to the normal censoring machinery can result in the development of tumors or autoimmunity. It is believed that censoring of autoimmune cells is abnormal in NOD mice and that apoptosis of autoreactive cells is restored by the establishment of mixed chimerism.

In addition to the preliminary data that NOD mice exhibit a relative radioresistance in vivo, and that cells from NOD mice are relatively radioresistant in vitro, a number of additional observations point to a defect in self-censoring via apoptosis. Lymphocytes from NOD mice are in a chronically more activated state (see preliminary data), are more readily activated by CD3 cross linking, but are less prone to undergo apoptosis. NOD mice produce an isoform of Fas Ligand (FasL) that is relatively inefficient in mediating clonal deletion by the Fas pathway (Kayagaki, N., et al., Proceed. Natl. Acad. Sci. USA, 94:3914 (1997)). This can be overcome by production of significantly increased levels of FasL. It is hypothesized that mixed chimerism restores effective apoptosis by providing a normally functional FasL molecule.

In general, autoimmunity develops due to a failure of the immune system to regulate lymphocytes with autoreactive repertoire. The immunological defects that lead to the development of diabetes are complex and may involve different components of the immune system that fail to either clonally eliminate or actively suppress autoreactive lymphocytes (Bach, J. F., et al., Immunol. Today, 16:353 (1995); Cameron, M. J., et al., Diabetes Metab. Rev., 14:177 (1998); Camitta, B. M., et al., N. Engl. J. Med.,, 306:712 (1982). The immunology of diabetes is characterized by a spontaneous loss of immunological tolerance to unique pancreatic β cell antigens. Loss of self-tolerance is manifested by the appearance of autoantibodies and T cells reactive to specific islet antigens (Bach, J. F., et al., Immunol. Today, 16:353 (1995); Katz, J. D., et al., Science, 268:1185 (1995); Naparstek, E., et al., Blood, 67:1395 (1986)). Failure to regulate these immunological responses to self-antigens results in infiltration of islets with mononuclear cells (insulitis) that eventually leads to the complete destruction of insulin-producing β cells (diabetes). Cumulative evidence in the literature suggests that T cells play a critical role in the development of diabetes. First, athymic nude NOD-nu/nu and severe combined immunodeficiency disease (NOD.scid) mice that lack T cells do not develop insulitis and diabetes (Yagi, H., et al., Eur. J. Immunol., 22:2387 (1992)). Second, antibodies to T cells, such as anti-CD3, inhibit the development of diabetes in NOD mice (Herold, K. C., et al., Diabetes, 41:385 (1992); Herold, K. C., et al., Diabetes, 36:796 (1987); Gerna, G., et al., J. Infectious Diseases, 164:488 (1991)). Third, the adoptive transfer of diabetogenic T cells into NOD or NOD.scid mice results in the early onset of diabetes (Haskins, K., et al., Science, 249:1433 (1990)). The nature of the immunological defects in NOD that fail to censor the autoreactive T cells are not well characterized and may be multifactorial. There is evidence, however, that a defect in the apoptotic process that fails to eliminate autoreactive T cells may play a dominant role in the development of diabetes in NOD. This contention is consistent with the observations that NOD mature T cells are resistant to apoptosis, NOD express a less-apoptotic allele of FasL, and in vitro treatment of diabetogenic splenocytes with FasL abrogates diabetes-induced by adoptive transfer (Kayagaki, N., et al., Proceed. Natl. Acad. Sci. USA, 94:3914 (1997); Lamhamedi-Cherradi, S. E., et al., Diabetologia, 41:178 (1998); Kim, S., et al., J. Immunol., 164:2931 (2000); Keman, N. A, et al., Blood, 68:770 (1986); Whitley, C. B., et al., Amer. J Medical Genetics, 46:209 (1993)). It is hypothesized that mixed chimerism prevents/treats diabetes by providing a functional form of FasL to eliminate autoreactive T cells.

### Example 5

### Determine How Chimerism Restores Effective Apoptosis in NOD Mice

The defective apoptosis in NOD may be because of Fas signaling or dysregulation of the anti-apoptotic machinery via the Bcl gene pathy, or a combination of the two. The interaction of Fas with FasL and selected anti- and pro-apoptotic genes such as Bcl-2 and Bax will be examined.

First it will be determined whether T cells from NOD mice at different ages and disease status are resistant to apoptosis using anti-Fas agonistic antibody (Jo2; PharMingen) to transduce the death signal. Splenic and lymphoid CD4⁺ and CD8⁺ T cells will be isolated by cell sorting. Cells will be incubated with 100 ng of Jo2 Ab with protein G (for cross-linking) for varying time points (4-24 hours). Apoptosis will be determined by flow cytometry using Annexin V-FITC and PI as early and late apoptotic markers, respectively. T cells from B10.BR and BALB/c will serve as controls. High levels of apoptosis in NOD T cells are anticipated if the defect is at the level of a functional FasL. Inasmuch as naïve T cells do not express Fas, significant apoptosis from control cells may not be detected. A second control in which T cells are stimulated with plate-bound anti-CD3 antibody to activate T cells will also be performed. NOD lymphocytes will be compared to normal controls. These cells will then be used for the apoptosis assay. A lack of apoptosis in NOD mice as compared with controls will suggest that the apoptotic defect in NOD is independent of FasL and may reside downstream to Fas-mediated signaling.

There is still considerable debate over whether the Fas/FasL and Bcl pathways for regulation of apoptotic cell death are independent or interrelated in biologic systems. It is hypothesized that there will be some overlap. The expression of anti-apoptotic Bcl-2 and pro-apoptotic Bax genes in anti-CD-3 activated NOD T cells will be examined to test whether the observed defect lies in dysregulated expression of these genes. Total RNA and proteins will be extracted form NOD and control cells and used for RT-PCR and Western blot analysis as described in the Preliminary Data section. An increase in the anti-apoptotic gene Bcl-2 with or without an obvious decrease in the pro-apoptotic gene Bax will imply that signaling downstream to Fas is important to the apoptosis-resistant phenotype of NOD cells. It is not anticipated that this will be the case since NOD autoreactive T cells have already been shown to be sensitive to wild type FasL-mediated apoptosis (Kim, S., et al., J. Immunol., 164:2931 (2000)). However, the fact that cells from NOD mice are relatively radioresistant may invoke more than one mechanism.

It will then be determined whether FasL expressed by NOD cells is efficient in causing apoptosis in Fas⁺ human Jurkat cells or A20 mouse thymoma cell line. Splenocytes from NOD mice and controls will be stimulated using ConA as a T cell mitogen for 24 hours. Activated T cells will be harvested by Ficoll gradient and used as effectors against Jurkat or A20 target cells that have been labeled with ³H-thymidine. Apoptosis will be determined by assessing the amount of radioactivity in the culture supernatant as described (Matsue, H., et al., Nat. Med., 5:930 (1999)). A lower level of apoptosis in cultures incubated with NOD T cells is anticipated as compared with controls if apoptosis is impaired. This will be consistent with the observation that NOD expresses a less apoptotic form of FasL (Kayagaki, N., et al., Proceed. Natl. Acad. Sci. USA, 94:3914 (1997)).

It will first be important to characterize the expression of anti and pro apoptotic gene expression in NOD mice as they progress from active insulitis and autoimmunity to full blown diabetes (EXPERIMENT 1). CD4⁺ and CD8⁺ lymphocytes from female NOD mice of different age groups and with different status of autoimmunity for the expression of Fas, FasL, Bcl-2, and Bax genes will be analyzed to determine whether there is a correlation between the expression of pattern of some of these genes and the development of diabetes. Splenocytes and bone marrow cells will be harvested from newborn NOD mice and mice at 1, 3, 4, 6, 8, 12, 16, 24, 32, and 48 weeks of age (n=4 per group). Age-matched female BALB/c and B10.BR mice will serve as controls. Mice will be monitored weekly for the development of glucosuria. At the time of euthanasia, the pancreata will be harvested and evaluated for severity of insulitis. As in previous experiments, the samples will be read in a blinded fashion. Spontaneous diabetes may develop as a result of dysregulation of the apoptotic machinery by time and escape of autoreactive clones. This may occur as autoreactive T cells early in life may be sensitive to apoptosis by FasL due to low level of anti-apoptotic and high levels of pro-apoptotic genes. If this contention holds, it is anticipated that the expression of the anti-apoptotic genes such as Bcl-2 will increase while the expression of pro-apoptotic genes, such as Bax, will decrease as a function of time and development of diabetes.

Experiment 1: Analysis of Anti- and Pro-Apoptotic Gene Expression In NOD.

| AGE | | | | | |
|---|---|---|---|---|---|
| (week) | N | FASL | FAS | BAX | BCL-2 |
| New born | 4 | RT-PCR/protein | Flow | RT-PCR/protein | RT-PCR/ |
| | | | | | protein |
| 1 | 4 | RT-PCR/protein | Flow | RT-PCR/protein | RT-PCR/ |
| | | | | | protein |
| 3 | 4 | RT-PCR/protein | Flow | RT-PCR/protein | RT-PCR/ |
| | | | | | protein |
| 4 | 4 | RT-PCR/protein | Flow | RT-PCR/protein | RT-PCR/ |
| | | | | | protein |
| 6 | 4 | RT-PCR/protein | Flow | RT-PCR/protein | RT-PCR/ |
| | | | | | protein |
| 8 | 4 | RT-PCR/protein | Flow | RT-PCR/protein | RT-PCR/ |
| | | | | | protein |
| 12 | 4 | RT-PCR/protein | Flow | RT-PCR/protein | RT-PCR/ |
| | | | | | protein |
| 16 | 4 | RT-PCR/protein | Flow | RT-PCR/protein | RT-PCR/ |
| | | | | | protein |

The expression of the genes that regulate apoptosis in mixed chimeras is analyzed to determine whether there is a correlation between the expression of some of these genes and the treatment of diabetes. These experiments will be designed based upon our findings from the prior studies. It is hypothesized that autoimmunity in NOD mice is abrogated by the elimination of autoreactive NOD T cells through apoptosis-mediated by donor cells in chimeras complementing the apoptotic machinery in NOD. Inasmuch as mixed chimerism cures diabetes, the apoptotic defect most probably resides at the level of FasL interaction with Fas, which is consistent with a less-apoptotic form of FasL expressed in NOD. To provide experimental evidence for this contention, the expression of Fas, FasL, Bcl-2, and Bax in NOD and B10.BR T cells in mixed chimeras will be analyzed at selected times after chimerism is established. The expression of these genes in sorted CD4⁺ and CD8⁺ T cells and APC (dendritic cells and macrophage) of donor and recipient origin harvested at 4, 8, and 12 weeks after chimerism is established will be analyzed by flow cytometry and RT-PCR. A correlation between the expression of FasL in B10.BR cells and the elimination of recipient autoreactive T cells is expected. A significant change in the levels of Bcl-2 and Bax in donor B10.BR is not anticipated nor NOD recipients if the FasL defect is the most dominant one in NOD.

The foregoing description is considered as illustrative only of the principles of the invention. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. Furthermore, since a number of modifications and changes will readily occur to those skilled in the art, it is not desired to limit the invention to the exact construction and process shown described above. Accordingly, all suitable modifications and equivalents may be resorted to falling within the scope of the invention as defined by the claims which follow.

The embodiments of the invention in which an exclusive property or privilege is claimed are defined as follows:

1. A method for treating a systemic autoimmune disease comprising:
subjecting said recipient to a composition that specifically depletes αβ-, and γδ-TCR⁺ T cells and/or CD8⁺ T cells in the recipient hematopoietic microenvironment, followed by transplantation with a donor cell preparation containing hematopoietic stem cells from a donor that are matched at the major histocompatibility complex class I K locus with the recipient hematopoietic microenvironment; and
grafting hematopoietic stem cells and/or tissue into said recipient.

2. The method of claim 1 in which said composition comprises antibodies specific for αβ-, and γδ-TCR⁺ T cells and/or CD8⁺ T cells.

3. The method of claim 1 in which said composition comprises antisense DNA that is directed against the precursors of αβ-, and γδ-TCR⁺ T cells and/or CD8⁺ T cells.

4. The method of claim 3 wherein antisense DNA alters the translation of the α-chain, β-chain, γ-chain, or δ-chain of TCR⁺ T cells.

5. The method of claim 3 wherein antisense DNA alters the transcription of the α-chain, β-chain, γ-chain, or δ-chain of TCR⁺ T cells.

6. The method of claim 1 in which said composition a cytotoxic drug specific for αβ-, and γδ-TCR⁺ T cells and/or CD8⁺ T cells.

7. The method of claim 1 wherein the recipient is further conditioned by subjecting the recipient to a total dose of total body irradiation of less than or equal to 300 cGy.

8. The method of claim 1 wherein the recipient is further conditioned by subjecting the recipient to an alkylating agent.

9. The method of claim 8 wherein said alkylating agent is cyclophosphamide.

10. The method of claim 1 wherein said composition specific to αβ-, and γδ-TCR⁺ T cells and/or CD8⁺ T cells in the recipient hematopoietic microenvironment totally eliminates said cells from the recipient hematopoietic microenvironment.

11. The method of claim 1, wherein said systemic autoimmune disease is type I diabetes and said graft comprises islet cells.

12. The method of claim 1, wherein said systemic autoimmune disease is lupus.

13. The method of claim 1, wherein said systemic autoimmune disease is rheumatoid arthritis.

14. The method of claim 1, wherein said systemic autoimmune disease is scleroderma.

15. The method of claim 1, wherein said systemic autoimmune disease is multiple sclerosis.

16. The method of claim 1, wherein said systemic autoimmune disease is Crohn's disease.

17. The method of claim 1, wherein said systemic autoimmune disease is Colitis.

18. A method for treating an autoimmune disease comprising subjecting said recipient treatment with a total dose of total body irradiation from 100 to 300 cGy, and treating the patient with a composition that specifically depletes αβ-, and γδ-TCR⁺ T cells and/or CD8⁺ T cells in the recipient hematopoietic microenvironment, followed by transplantation with a donor cell preparation containing hematopoietic stem cells from a donor that are matched at the major histocompatibility complex class I K locus with the recipient hematopoietic microenvironment; and
introducing a graft necessary to complement said recipient's disease state.

19. The method of claim 18, wherein the recipient is further treated with an alkylating agent before, during, or after exposure to said composition that specifically depletes αβ-, and γδ-TCR⁺ T cells and/or CD8⁺ T cells in the recipient hematopoietic microenvironment.

20. The method of claim 19, wherein said alkylating agent is cyclophosphamide.

21. A method of partially or completely reconstituting a mammal's lymphohematopoietic system comprising administering to the mammal a composition that specifically depletes αβ, and γδ-TCR⁺ T cells and/or CD8⁺ T cells in the recipient hematopoietic microenvironment, followed by transplantation with a donor cell preparation containing hematopoietic stem cells from a donor that are matched at the major histocompatibility complex class I K locus with the recipient hematopoietic microenvironment.

22. The method of claim 21, in which the mammal suffers from autoimmunity.

23. The method of claim 22, in which the autoimmunity is diabetes.

25. The method of claim 22, in which the autoimmunity is multiple sclerosis.

25. The method of claim 22, in which the autoimmunity is sickle cell.

26. The method of claim 22, in which the autoimmunity is anemia.

27. The method of claim 22, in which the mammal suffers from a hematologic malignancy.

27. The method of claim 21, in which the mammal requires a solid organ or cellular transplant.

28. The method of claim 21, in which the mammal suffers from immunodeficiency.
